(19) United States
(12) Patent Application Publication
(10) Pub. No.: US 2025/0292868 A1
(43) Pub. Date: Sep. 18, 2025

(54) SYSTEMS AND METHODS FOR MULTIMODAL CONVERSATIONAL AGENTS FOR BIOLOGICAL SEQUENCE ANALYSIS

(71) Applicants: InstaDeep Ltd, London (GB); BioNTech SE, Mainz (DE)

(72) Inventors: Thomas Pierrot, Boston, MA (US); Bernardo P. De Almeida, Paris (FR); Guillaume Richard, Paris (FR); Hugo Dalla-Torre, Paris (FR); Alexandre Laterre, London (GB); Karim Beguir, London (GB); Lorenz Johann Leopold Hexemer, Nierstein (DE); Stefan Jean Yvon Laurent, Cologne (DE); Maren Lang, Mainz (DE); Priyanka Pandey, Mainz (DE); Ugur Sahin, Mainz (DE)

(21) Appl. No.: 19/075,312

(22) Filed: Mar. 10, 2025

Related U.S. Application Data
(60) Provisional application No. 63/566,768, filed on Mar. 18, 2024.
(60) Provisional application No. 63/683,672, filed on Aug. 15, 2024.

(51) Int. Cl. G16B 30/00; G06N 3/0455; G16B 40/30

(52) U.S. Cl. CPC G16B 30/00; G06N 3/0455; G16B 40/30

(57) ABSTRACT

Provided herein are technologies for framing and evaluating biological sequence-based analysis tasks in a unified, natural-language-based, text in and text out format. Among other things, methods and systems of the present disclosure provide machine-learning technologies for combining biological sequence data, representing, for example, DNA, RNA, and protein sequences, with natural language, conversational style prompts that set out particular analysis tasks to be performed on the biological sequence data. This approach, for example, allows complex analysis tasks, including, but not limited to, identification of various sequence modifications, genes, and regulatory elements in DNA sequences, and quantification of properties such as degradation propensity of RNA and protein stability, to be input to a machine learning model in a uniform text-based format and for output to be generated in a same, unified, text-based format.

### CROSS-REFERENCE TO RELATED APPLICATIONS

This application claims priority to U.S. Provisional Patent Application No. 63/566,768, filed Mar. 18, 2024, the title of which is “Multi-modal agent for genomics,” and to U.S. Provisional Patent Application No. 63/683,672, filed Aug. 15, 2024, the title of which is “Systems and methods for multimodal conversational agents for biological sequence analysis”, the content of each of which is incorporated herein by reference in its entirety.

### BACKGROUND

Despite significant efforts to apply machine learning technologies to healthcare, pharmaceutical, and various biomedical research fields, they remain challenging to implement and utilize without expertise in coding. Moreover, conventional implementations focus on creating individual models tailored or fine-tuned to perform single, isolated, specific tasks. As a result, there has been a proliferation of individual models, each suited of a particular task. Accordingly, modeling multitudes of different interactions that take place in various biological pathways with individualized and bespoke models can be a daunting task.

### SUMMARY

Provided herein are technologies for framing and evaluating biological sequence-based analysis tasks in a unified, natural-language-based, text-in and text-out format. Among other things, methods and systems of the present disclosure provide machine-learning technologies for combining biological sequence data, representing, for example, DNA, RNA, and protein sequences, with natural language, conversational style prompts that set out particular analysis tasks to be performed on the biological sequence data. This approach, for example, allows complex analysis tasks, including, but not limited to, identification of various sequence modifications, genes, and regulatory elements in DNA sequences, and quantification of properties such as degradation propensity of RNA and protein stability, to be input to a machine learning model in a uniform text-based format and for output to be generated in a same, unified, text-based format.

Not only does this approach provide a convenient conversational style input-output framework that facilities user interaction with the underlying machine learning-based technology, but, moreover, it provides a unified and modular platform that can readily incorporate a wide variety of input types and facilitates transfer learning between tasks, leading to improvements in training efficiency and accuracy. In particular, as described in further detail herein, technologies of the present disclosure allow all desired analysis tasks to be expressed with a same vocabulary, such as a concatenation of natural language (e.g., English) and biological sequence (e.g., DNA) vocabularies, and to learn to solve them by minimizing a unified objective, allowing for seamless new task integration and generalization, as well as extension to various biological data modalities (e.g. sequencing experiments, imaging).

In one aspect, the present disclosure provides methods for evaluating multiple biological sequence-based tasks via combined natural language and biological sequence-based queries, said methods comprising: (a) receiving and/or accessing, by a processor of a computing device, (i) a natural language prompt and (ii) biological sequence data representing one or more biological sequences for evaluation (e.g., a DNA sequence, an RNA sequence, a protein sequence); (b) generating, by the processor, using a biological language encoder, one or more (e.g., a plurality of) biological sequence embeddings based on the biological sequence data; (c) determining, by the processor, (e.g., using a natural language encoder) one or more text embeddings based on the natural language prompt; (d) generating, by the processor, using a natural language decoder, a natural language response based on (i) the one or more text embeddings and (ii) the one or more biological sequence embeddings; and (e) storing and/or providing, by the processor, the determined natural language response for display and/or further processing.

In some embodiments, biological sequence data is or comprises deoxyribonucleic acid (DNA) sequence data representing one or more nucleotide sequence(s).

In some embodiments, biological sequence data is or comprises ribonucleic acid (RNA) sequence data representing one or more RNA sequence(s) (e.g., sequences of ribonucleotides).

In some embodiments, biological sequence data is or comprises polypeptide sequence data representing one or more polypeptide sequence(s) (e.g., protein sequence(s)).

In some embodiments, biological sequence data is or comprises one or more sequence representation(s) of a first type (e.g., RNA sequence data; e.g., protein sequence data) and the method comprises converting, by the processor, the one or more sequence representations of the first type to one or more corresponding sequence representations of a second type (e.g., DNA sequences) for use as input to the biological language encoder

In some embodiments, a biological language encoder model is or has been trained using a training dataset comprising a plurality of example biological sequences of the second type (e.g., but not the first type).

In some embodiments, a biological sequence encoder receives, as input, one or more sequences of tokens, each sequence of tokens representing at least a portion of the one or more biological sequences.

In some embodiments, a biological sequence encoder model generates, the one or biological sequence embeddings based on the one or more sequences of tokens received as input.

In some embodiments, one or more biological sequence embeddings are or comprise one or more sets of biological sequence embedding vectors, each set of biological sequence embedding vectors (i) corresponding to and generated based on a particular sequence of tokens received as input and (ii) comprising for each token of the particular sequence, a corresponding embedding vector.

In some embodiments, provided methods comprise: generating, by the processor, from the one or more biological sequence embeddings, one or more corresponding projected embeddings, wherein the biological sequence embeddings have a first dimensionality and the corresponding projected embeddings have a second dimensionality, different from the first and matching a dimensionality of the one or more text embeddings; and using the one or more projected embeddings and the one or more text embeddings as input to the natural language decoder.

In some embodiments, provided methods comprise using a projection model to generate the one or more projected embeddings, wherein the projection model receives, as input the one or more biological sequence embeddings and generates, as output the one or more corresponding projected embeddings.

In some embodiments, a projection model comprises one or more cross attention layers.

In some embodiments, a projection model receives the one or more text embeddings as input, thereby generating the one or more projected embeddings based on the biological sequence embeddings and the text embeddings (e.g., wherein the projection model comprises at least two cross-attention layers, a first that receives the one or more biological sequence embeddings as input and a second that receives the one or more text embeddings as input).

In some embodiments, a natural language decoder model is or comprises a pre-trained model, having been trained using a training corpus comprising a plurality of natural language text.

In some embodiments, a natural language prompt comprises one or more positional sequence tags, each identifying a particular one of the one or more biological sequences and a corresponding position within the natural language prompt, and provided methods comprise: inserting the one or more biological sequence embeddings and/or projections thereof within the one or more text embeddings based on their corresponding positions as identified via the one or more positional sequence tags to create a combined embedding; and using the combined embedding as input to the natural language decoder model.

In some embodiments, provided methods comprise: generating, by the processor, using a trained projection model, from the one or more biological sequence embeddings, one or more corresponding projected embeddings having a dimensionality matching that of the one or more text embeddings (e.g., wherein the biological sequence embeddings have a first dimensionality and the corresponding projected embeddings have a second dimensionality, different from the first and matching a dimensionality of the one or more text embeddings), said trained projection model having been trained using a natural language question and answer dataset comprising a plurality of example natural language prompts and corresponding natural language answers; and using the one or more projected embeddings and the one or more text embeddings as input to the natural language decoder.

In some embodiments, a biological sequence encoder is a pre-trained and subsequently fine-tuned model, having (i) been initially pre-trained (e.g., separately from the projection model and natural language decoder) in an unsupervised fashion using a biological sequence training dataset comprising a plurality of example biological sequences, and (ii) subsequently, trained in tandem with the projection model, using the natural language question and answer dataset.

In some embodiments, a natural language decoder is a pre-trained model, having been trained (e.g., separately from the projection model and the biological sequence encoder) using a training corpus comprising a plurality of natural language text (e.g., in an unsupervised fashion).

In some embodiments, provided methods comprise: prior to step (a), causing, by the processor, display of a graphical user interface (GUI) comprising a textual input widget for user entry of free-form text; at step (a), receiving, by the processor, via the textual input widget, as the natural language prompt; user input of text; and at step (d) causing, by the processor, display of the determined natural language response.

In some embodiments, a GUI is or comprises a chatbot graphical dialog (i) comprising the textual input widget and (ii) in which the determined natural language response is displayed.

In some aspects, the present disclosure provides methods for evaluating multiple tasks relating to and accommodating one or more biological input modalities via unified natural language-based query and response interface, the method comprising: (a) receiving and/or accessing, by a processor of a computing device, (i) a natural language prompt and (ii) biological object data representing a biological object for evaluation, wherein the biological object data is a particular one of a set of possible datatypes, each associated with a particular biological object encoder of a multi-modal machine learning model; (b) determining and selecting, by the processor, a particular biological object encoder associated with the particular datatype of the biological object data, and generating, by the processor, using the selected biological object encoder, one or more (e.g., a plurality of) biological object embeddings based on the biological object data; (c) determining, by the processor, (e.g., using a natural language encoder) one or more text embeddings based on the natural language prompt; (d) generating, by the processor, using a natural language decoder, a natural language response based on (i) the one or more text embeddings and (ii) the one or more biological objecting embeddings; and (e) storing and/or providing, by the processor, the determined natural language response for display and/or further processing.

In some embodiments, a set of possible datatypes comprises one or more types of biological sequence data, each corresponding to and representing a particular type of biological sequence (e.g., a DNA sequence; e.g., an RNA sequence; e.g., a polypeptide sequence) and the multi-modal machine learning model comprises at least one biological language encoder having been trained via a biological sequence training dataset comprising a plurality of example biological sequences.

In some embodiments, a multi-modal machine learning model comprises a multi-omic biological language encoder having been trained via a biological sequence training dataset comprising a plurality of example biological sequences of at least two distinct types [e.g., DNA sequences and RNA sequences; e.g., (i) DNA sequences and/or RNA sequences and (ii) polypeptide sequences].

In some embodiments, a multi-modal machine learning model comprises a plurality of biological language models, each corresponding to a particular type of biological sequence and having been trained on a dataset comprising a plurality of sequences of the corresponding type.

In some embodiments, a set of possible datatypes comprises one or more types of biological structure models representing 3D structure of biological molecules (e.g., 3D DNA structure, e.g., 3D RNA structure, e.g., 3D protein structure).

In some aspects, the present disclosure provides systems for evaluating multiple biological sequence-based tasks via combined natural language and biological sequence-based queries, said provided systems comprising: a processor of a computing device; and memory having instructions stored thereon, wherein the instructions, when executed by the processor, cause the processor to: (a) receive and/or access (i) a natural language prompt and (ii) biological sequence data representing one or more biological sequences for evaluation (e.g., a DNA sequence, an RNA sequence, a protein sequence); (b) generate, using a biological language encoder, one or more (e.g., a plurality of) biological sequence embeddings based on the biological sequence data; (c) determine (e.g., using a natural language encoder) one or more text embeddings based on the natural language prompt; (d) generate, using a natural language decoder, a natural language response based on (i) the one or more text embeddings and (ii) the one or more biological sequence embeddings; and (e) store and/or provide the determined natural language response for display and/or further processing.

In some aspects, the present disclosure provides systems for evaluating multiple tasks relating to and accommodating one or more biological input modalities via unified natural language-based query and response interface, said provided systems comprising: a processor of a computing device; and memory having instructions stored thereon, wherein the instructions, when executed by the processor, cause the processor to: (a) receive and/or access (i) a natural language prompt and (ii) biological object data representing a biological object for evaluation, wherein the biological object data is a particular one of a set of possible datatypes, each associated with a particular biological object encoder of a multi-modal machine learning model; (b) determine and/or select a particular biological object encoder associated with the particular datatype of the biological object data, and generate, using the selected biological object encoder, one or more (e.g., a plurality of) biological object embeddings based on the biological object data; (c) determine (e.g., using a natural language encoder) one or more text embeddings based on the natural language prompt; (d) generate, using a natural language decoder, a natural language response based on (i) the one or more text embeddings and (ii) the one or more biological objecting embeddings; and (e) store and/or provide the determined natural language response for display and/or further processing.

In some aspects, the present disclosure provides computer-implemented methods comprising: receiving a natural language prompt referring to a nucleotide sequence; determining, from the prompt, a sequence of input tokens comprising a placeholder token for the nucleotide sequence; processing the sequence of input tokens using a language encoder of a multi-modal model to generate a sequence of language embedding vectors; obtaining data indicative of a sequence of nucleotides; processing the data indicative of the sequence of nucleotides using a nucleotide encoder of the multi-modal model to generate a sequence of nucleotide embedding vectors; combining the sequence of nucleotide embedding vectors with the sequence of language embedding vectors using the placeholder token to generate a mixed sequence of embedding vectors; and processing the mixed sequence of embedding vectors using a language decoder of the multi-modal model to generate a response to the prompt, the response comprising the sequence of output tokens.

In certain embodiments, a prompt is received via a user interface, the method comprising outputting, via the user interface, the response to the prompt.

In certain embodiments, combining a sequence of nucleotide embedding vectors with a sequence of language embedding vectors comprises inserting the sequence of nucleotide embedding vectors into the sequence of language embedding vectors at a position indicated by the placeholder token.

In certain embodiments, a prompt is indicative of a classification task with an associated plurality of classes, the sequence of output tokens is indicative of a first class of the plurality of classes, and provided methods comprise: generating, using the multi-modal model, a plurality of sequences of output tokens in response to the prompt; sampling, from the plurality of sequences of output tokens, one or more sequences of output tokens indicative of each class of the plurality of classes; computing, for the or each sequence of output tokens indicative of each class, a respective perplexity of the multimodal model; and determining, based at least in part on the computed perplexities, a value indicative of a confidence of the multi-modal model in the first class being correct.

In certain embodiments, provided methods comprise outputting, with the response to the prompt, the value indicative of the confidence of the multi-modal model.

In certain embodiments, determining a value indictive of the confidence of the multi-modal model comprises processing the respective perplexities using a calibration model.

In certain embodiments, provided methods comprise determining a proportion of the plurality of sequence of output tokens that are indicative of the first class; and training the calibration model to determine the value indicative of the confidence of the multi-modal model to correspond to the proportion of the plurality of sequence of output tokens that are indicative of the first class.

In certain embodiments, a classification task is a binary classification task.

In certain embodiments, provided methods comprise: obtaining a target sequence of output tokens; and updating parameter values of the multi-modal model using instruction tuning based at least in part on the sequence of output tokens and the target sequence of output tokens.

In certain embodiments, provided methods comprise freezing the language decoder during the updating of the parameter values of the multi-modal model.

In certain embodiments, generating a sequence of nucleotide embedding vectors comprises: generating a sequence of intermediate nucleotide embedding vectors as an output of the nucleotide encoder; and projecting, using a projection model, the sequence of intermediate nucleotide embedding vectors into an embedding space of the language embedding vectors.

In certain embodiments, a projection model comprises a resampling model, wherein the resampling model uses cross-attention between the intermediate nucleotide embedding vectors and a set of learnable queries.

In certain embodiments, a resampling model further uses cross-attention between the sequence of language embedding vectors and the set of learnable queries.

In certain embodiments, projecting a sequence of intermediate nucleotide embedding vectors into an embedding space of the language embedding vectors comprises processing the sequence of language embedding vectors and the sequence of intermediate nucleotide embedding vectors using the projection model.

In certain embodiments, a sequence of nucleotide embedding vectors has a predetermined length that is different from a length of the sequence of intermediate nucleotide embedding vectors.

In certain embodiments, a predetermined length is between 10 and 100 nucleotide embedding vectors.

In certain embodiments, a predetermined length is 64 nucleotide embedding vectors.

In certain embodiments: language embedding vectors and nucleotide embedding vectors have a first number of dimensions; and intermediate nucleotide embedding vectors have a second number of dimensions different to the first number of dimensions.

In certain embodiments, a prompt comprises a file indicator, and provided methods comprise: retrieving, from a database, a data file corresponding to the file indicator, the data file comprising the data indicative of the sequence of nucleotides; and tokenizing the prompt to generate the sequence of input tokens, including introducing the placeholder token in place of the file indicator.

In certain embodiments: a placeholder token is a first placeholder token, the file indicator is a first file indicator, the data file is a first data file, the sequence of nucleotides is a first sequence of nucleotides, and the sequence of nucleotide embedding vectors is a first sequence of nucleotide embedding vectors; a prompt comprises a second file indicator; and tokenizing a prompt includes introducing a second placeholder token in place of the second file indicator; and provided methods comprise: retrieving a second data file corresponding to the second file indicator, the second data file comprising data indicative of a second sequence of nucleotides; processing the data indicative of the second sequence of nucleotides to generate a second sequence of nucleotide embedding vectors; and generating the mixed sequence of embedding vectors comprises combining the second sequence of nucleotide embedding vectors with the sequence of language embedding vectors and the first sequence of nucleotide embedding vectors using the first placeholder token and the second placeholder token.

In certain embodiments, generating the mixed sequence of embedding vectors comprises inserting the second sequence of nucleotide embedding vectors into the sequence of language embedding vectors at a position indicated by the second placeholder token.

In certain embodiments: a nucleotide encoder is a first nucleotide encoder; and processing data indicative of the second sequence of nucleotides uses a second nucleotide encoder that is different from the first nucleotide encoder.

In certain embodiments, data within the second data file has a different modality to the data within the first data file.

In certain embodiments, data within the second data file comprises image data.

In certain embodiments: data indicative of the sequence of nucleotides comprises a sequence of nucleotide tokens; and a nucleotide encoder model comprises a transformer encoder.

In some aspects, the present disclosure provides systems comprising: one or more processors; and one or more non-transitory computer-readable media storing: a multimodal model comprising a language encoder, a nucleotide encoder, and a language decoder; and instructions which, when executed by the one or more processors, cause the one or more processors to carry out one or more provided methods described herein (e.g., in paragraphs above).

In some aspects, the present disclosure provides a computer program product comprising instructions which, when the program is executed by a computer, cause the computer to carry out one or more provided methods described herein (e.g., in paragraphs above).

In certain embodiments, a computer program product comprises one or more non-transitory computer-readable media storing the instructions.

Features of embodiments described with respect to one aspect of the invention may be applied with respect to another aspect of the invention.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1 is a block flow diagram of a method for evaluating multiple biological-sequence based tasks via combined natural language and biological sequence-based queries, according to an illustrative embodiment.

FIG. 2A is a schematic illustrating input queries and output responses generated via multi-modal machine learning technologies described herein, according to an illustrative embodiment.

FIG. 2B is a schematic illustrating a machine learning model architecture for accommodating biological sequence data in combination with natural language text-based prompts and generating natural-language responses, according to an illustrative embodiment.

FIG. 2C is a schematic illustrating a training framework of a machine learning model architecture to receive, as input, biological sequence data in which a biological sequence is represented via a sequence of tokens and predict values of unseen or masked tokens, according to an illustrative embodiment.

FIG. 3 is a block flow diagram of a process for evaluating and combining biological sequence data with a natural language prompt to generate a response, according to an illustrative embodiment.

FIG. 4 is a schematic showing an architecture for handling multiple biological object datatypes, according to an illustrative embodiment.

FIG. 5A is a schematic diagram showing a system used in certain embodiments described herein.

FIG. 5B is a schematic of a non-transitory storage medium used in certain embodiments described herein.

FIG. 6 is a block diagram of an exemplary cloud computing environment used in certain embodiments.

FIG. 7 is a block diagram of an example computing device and an example mobile computing device used in certain embodiments.

FIG. 8A is a schematic illustrating a conversational agent, referred to as ChatNT, that can be prompted to solve a variety of biological tasks and downstream tasks, according to an illustrative embodiment.

FIG. 8B is a bar graph providing statistics about number of English and DNA tokens available for each task in a genomics instructions dataset, according to an illustrative embodiment. English question/answer instructions are tokenized with the LLaMA tokenizer while DNA sequences are tokenized using the Nucleotide Transformer tokenizer.

FIG. 8C is a schematic illustrating conversational agent ChatNT approach for building a multimodal and multi-task genomics AI system, according to an illustrative embodiment. ChatNT can be prompted in English to solve various tasks given an input question and nucleotide sequence. A user inputs a DNA sequence (fasta file) and asks the agent to evaluate the degradation rate of the given RNA sequence. The question tokens are combined with the projected DNA representations before passing through the English Language Model decoder. A pretrained decoder writes the answer through next-token prediction, in this case predicting the degradation rate of the input sequence.

FIG. 9 is an illustration showing examples of ChatNT conversations on DNA, RNA and Protein tasks. For each conversation, a question from a user and an answer of the ChatNT agent are both shown. The projected embeddings of the input DNA sequences are incorporated in the question at the position of @myseq.fna.

FIG. 10A is a schematic of a projection model without cross-attention to text embeddings, according to an illustrative embodiment.

FIG. 10B is a schematic of a projection model with cross-attention to text embeddings, according to an illustrative embodiment.

FIG. 10C is a radar plot showing MCC performance per task for two projections of ChatNT on 18 textualized tasks of Nucleotide Transformer benchmark, according to an illustrative embodiment.

FIG. 10D is a bar plot showing average MCC over 18 tasks and the standard error of the mean average for two projections of ChatNT, according to an illustrative embodiment.

FIG. 11A is a bar plot showing average performance of ChatNT, ChatNT with no English-aware projection, and 13 different genomics foundation models across all 18 tasks of the Nucleotide Transformer benchmark, according to an illustrative embodiment. Bar-plots display the mean MCC over all tasks and the standard error of the mean.

FIG. 11B is a radar plot depicting ChatNT performance for 18 tasks compared with specialized NTv2 models fine-tuned individually for each task, according to an illustrative embodiment.

FIG. 12 is a grid plot showing performance of ChatNT, ChatNT with no English-aware projection, and 13 different foundation models on the 18 tasks from the Nucleotide Transformer benchmark, according to an illustrative embodiment.

FIG. 13A shows an example of prediction performance and conversations for a biological task: a conversation for a classification task is on the left and a heatmap displaying a confusion matrix comparing the predicted labels of ChatNT and observed labels, reporting a performance metrics, is on the right.

FIG. 13B shows an example of prediction performance and conversations for a biological task: a conversation for a regression task is on the left and a scatter plot comparing predictions of ChatNT and observed values, reporting Pearson correlation coefficient, is on the right.

FIG. 13C shows an example of prediction performance and conversations for a biological task: a conversation for a regression task is on the left and a scatter plot comparing predictions of ChatNT and observed values, reporting Pearson correlation coefficient, is on the right.

FIG. 13D shows an example of prediction performance and conversations for a biological task: a conversation for a classification task is on the left and a heatmap displaying a confusion matrix comparing the predicted labels of ChatNT and observed labels, reporting a performance metrics, is on the right.

FIG. 13E shows an example of prediction performance and conversations for a biological task: a conversation for a classification task is on the left and a heatmap displaying a confusion matrix comparing the predicted labels of ChatNT and observed labels, reporting a performance metrics, is on the right.

FIG. 13F shows an example of prediction performance and conversations for a biological task: a conversation for a regression task is on the left and a scatter plot comparing predictions of ChatNT and observed values, reporting Pearson correlation coefficient, is on the right.

FIG. 14 shows examples of conversations included in ChatNT training data for different genomics tasks. For each conversation a question from a user and an answer of the agent are both shown. The projected embeddings of input DNA sequences are incorporated in the question at the position of @myseq.fna.

FIG. 15 shows examples of conversations included in ChatNT training data for different RNA tasks, using a respective complementary DNA sequence. For each conversation a question from a user and an answer of the agent are both shown. The projected embeddings of input RNA sequences are incorporated in the question at the position of @myseq.fna.

FIG. 16 shows examples of conversations included in ChatNT training data for different protein tasks, using the respective coding sequences (CDS) (i.e., DNA or RNA). For each conversation a question from a user and an answer of the agent are both shown. The projected embeddings of input CDS sequences are incorporated in the question at the position of @myseq.fna.

FIG. 17A is a bar plot showing performance of ChatNT compared with respective baselines per task, according to an illustrative embodiment. A metric used for each task is the same used in the respective baseline study.

FIG. 17B is a violin plot showing a comparison between ChatNT and baselines for all tasks with the same metrics as in FIG. 12A, according to an illustrative embodiment.

FIG. 17C is a violin plot showing a comparison between ChatNT and baselines for classification tasks with the same metrics as in FIG. 12A, according to an illustrative embodiment.

FIG. 17D is a violin plot showing a comparison between ChatNT and baselines for regression tasks with the same metrics as in FIG. 12A, according to an illustrative embodiment.

FIG. 18A is a block flow diagram describing a perplexity-based classifier based on ChatNT answers, according to an illustrative embodiment.

FIG. 18B is a calibration plot for a task of human chromatin accessibility (cell line HepG2) comparing the predicted probability and fraction of positives over ten bins for the original and calibrated perplexity-base classifiers, according to an illustrative embodiment.

FIG. 18C is a histogram showing a predicted probability over ten bins for original perplexity-base classifiers, according to an illustrative embodiment.

FIG. 18D is a histogram showing a predicted probability over ten bins for calibrated perplexity-base classifiers, according to an illustrative embodiment.

FIG. 18E is a bar plot comparing performance (MCC) of ChatNT answers (yes vs no) and associated derived perplexity-based probabilities for all binary classification tasks, according to an illustrative embodiment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Certain Definitions

About or Approximately: The term “about” or “approximately”, when used herein in reference to a value, refers to a value that is similar to the referenced value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Biological sequence: As used herein, the term “biological sequence” refers to a physical sequence of biological building blocks (e.g., nucleotides, amino acids, etc.) typically forming a biopolymer, such as DNA, RNA, and polypeptides (e.g., proteins and/or peptides). In certain embodiments, a biological sequence is or comprises a nucleotide sequence. For example, a biological sequence may be a DNA sequence. For example, a biological sequence may be an RNA sequence. In certain embodiments, a biological sequence may be a sequence of amino acids, such as a polypeptide sequence (e.g., a protein sequence; e.g., a peptide sequence).

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. In some embodiments, cancer may be characterized by a solid tumor. In some embodiments, cancer may be characterized by a hematologic tumor. In general, examples of different types of cancers known in the art include, for example, triple negative breast cancer (TNBC), hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, ovarian cancer, breast cancer, glioblastomas, colorectal cancer, gastro-intestinal cancers and nervous system cancers, benign lesions such as papillomas, and the like.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of one or more named elements or steps” also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: In some embodiments, the methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Machine learning module, machine learning model: As used herein, the terms “machine learning module” and “machine learning model” are used interchangeably and refer to a computer implemented process (e.g., a software function) that implements one or more particular machine learning algorithms, such as an artificial neural networks (ANNs), random forest, decision trees, support vector machines, and the like, in order to determine, for a given input, one or more output values. In certain embodiments, machine learning models are deep learning models or deep neural networks—for example, ANNs that comprise, in addition to an input layer and an output layer, one or more hidden layers (e.g., in between). Examples of deep learning models include, without limitation, recurrent neural networks (RNNs) (e.g., long short-term memory networks (LSTMs), bi-directional LSTMs (biLSTMs)), attention-based networks, such as transformer models, and convolutional neural networks (CNNs). In some embodiments, machine learning modules implementing machine learning techniques are trained in a supervised manner, for example using curated and/or manually annotated datasets. In certain embodiments, machine learning models may be trained in an unsupervised manner, using unlabeled data. In certain embodiments, a machine learning model may be trained via a reinforcement approach, for example wherein a reward/penalty system is used to train a machine learning model to learn strategies for accomplishing specified tasks. Training a machine learning model may be used to determine various parameters of a model, such as weights associated with layers in neural networks. In some embodiments, once a machine learning module is trained, e.g., to accomplish a specific task, such as predicting types of hidden nucleotides within of nucleotide sequences (e.g., DNA sequences) based on their context, values of determined parameters are fixed and the machine learning module is used to process new data (e.g., different from the training data), such as a new nucleotide sequence. The process of presenting a machine learning model with multiple examples, comparing its output to known, ground truth values, and updating parameters to progressively improve performance may be referred to as training, while the use of a (e.g., previously trained) machine learning model to generate predictions about new data, for which ground truth values may be unknown, may be referred to as inference. In some embodiments, machine learning modules may receive feedback, e.g., based on user review of accuracy, and such feedback may be used as additional training data, for example to dynamically update the machine learning module. In some embodiments, a trained machine learning module is a classification algorithm with adjustable and/or fixed (e.g., locked) parameters, e.g., a random forest classifier. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC), field programmable gate arrays (FPGAs), and the like).

Gene and gene element(s): The term “gene”, as used herein, refers to a series of nucleotides in a DNA sequence that is transcribed into a functional RNA (e.g., encoding a specific protein and/or portions thereof). The term “gene elements”, as used herein, refers to those portions of nucleotide sequences that are synthesized to create proteins and/or portions thereof (e.g., protein segments). In certain embodiments, gene elements contrast with regulatory elements that do not code for proteins, but, rather, are collections of nucleotides that, for example, impact expression of genes. Gene elements include, without limitation, protein-coding genes, lncRNAs, 5′UTRs, 3′UTRs, exons, introns, splice acceptors and donor sites.

Genomic element(s): As used herein, the term “genomic elements” refers to subunits of nucleotide sequences, which may be known, determined, or predicted to perform particular functions, such as coding for proteins and/or controlling gene expression. Genomic elements include, for example, gene elements and regulatory elements.

Natural language: As used herein, the term “natural language” refers to language of ordinary speaking and writing, such as English, Mandarin, Hindi, Spanish, French, Arabic (e.g., modem standard Arabic; e.g., Egyptian Arabic), Bengali, Portuguese, Russian, Urdu, Indonesian, German, Japanese, Pidgin, Marathi, Telugu, Turkish, Tamil, Yue Chinese, Vietnamese, Wu Chinese, Tagalog, Korean, Persian, etc.

Nucleic acid: As used herein, the term “nucleic acid” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleotides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g; phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is an mRNA. In some embodiments where an RNA is an mRNA, an RNA typically comprises at its 3′ end a poly(A) region. In some embodiments where an RNA is an mRNA, an RNA typically comprises at its 5′ end an art-recognized cap structure, e.g., for recognizing and attachment of an mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Regulatory element(s): The term “regulatory elements”, as used herein, refer to portions of nucleotide sequences (e.g., non-coding portions) that regulate gene expression (e.g., transcription of neighboring genes). Regulatory elements include, without limitation, polyA signals, tissue-invariant and tissue-specific promoters and enhancers, and CTCF-bound sites.

Sequence data: The term “sequence data”, as used herein, refers to a (e.g., computer) representation of a biological sequence. Sequence data may represent structural components, or building blocks, of a biological sequence in a variety of forms, such as a series of alpha numeric characters, a series of tokens, a set of one-hot encodings, and the like. In certain embodiments, orders of characters, tokens, one-hot vectors etc., may encode and/or reflect relative positions of corresponding sub-units within a biological sequence. For example, biological sequence data may be nucleotide sequence data or polypeptide sequence data. For example, in certain embodiments, nucleotide sequence data represents a nucleotide sequence, such as a polynucleotide or DNA sequence. In certain embodiments, polypeptide sequence data represents a polypeptide (e.g., protein) sequence.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

Methods and systems of the present disclosure allow for natural language-based (e.g., text) prompts to be combined with biological sequence data in order to frame a variety of sequence analysis tasks in a unified text-based format. Among other things, this approach allows for creation of a multi-modal and multi-task machine learning model that is capable of interacting with natural language input as well as one or more types of biological sequences, and performing a plurality of analysis tasks, ranging from identification and localization of particular genomic elements—such as genes and various regulatory elements—to determining quantitative predictions of properties such as stability, melting point, protein fluorescence, and the like. Not only does this framework provide a convenient interface with which a user can interact to query a machine learning model, but, among other things, the unified input/output format as described herein facilitates multi-task training, allowing machine learning models of the present disclosure to benefit from transfer learning, improving training efficiency and ultimate performance of resultant trained models. Additionally or alternatively, the present disclosure provides a flexible architecture that can be used to accommodate a variety of biological data modalities, and interleave them with natural language prompts that can be used to specify tasks to be performed, as well as provide metadata about biological inputs (e.g., type of input data, organism, experiment format, etc.). For example, while use of biological sequence data—such as DNA, RNA, and protein sequences—are described and demonstrated here, e.g., in the Example, other modalities, such as analysis of sequencing experiments, imaging, 3D structural data, and the like, along with relevant tasks.

### A. Machine Learning Technologies for Unified Analysis of Natural Language Prompts and Biological Sequence Data

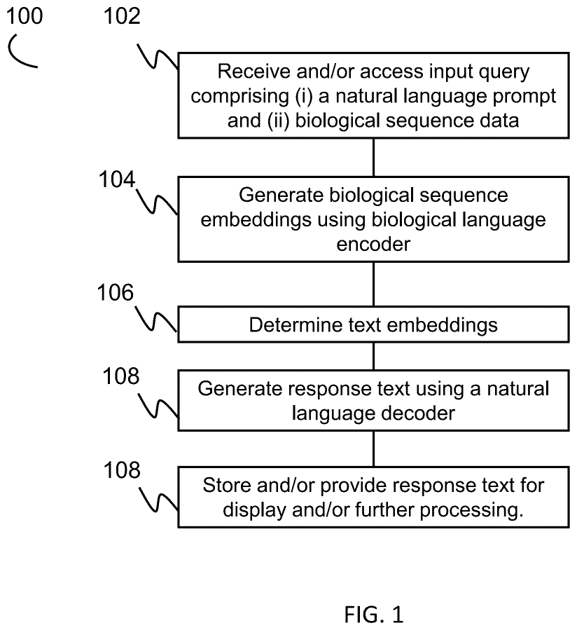

As shown in FIG. 1, in an example process 100, methods and systems of the present disclosure may operate on an input query comprising a natural language prompt and biological sequence data that is received and/or accessed 102 by a processor of a computing device.

In certain embodiments, a natural language prompt is or comprises text representing a question, instruction, etc., phrased in a natural language, such as English, French, Thai, etc. In certain embodiments, a natural language prompt is provided in a single language. In certain embodiments, multiple natural languages may be used.

In certain embodiments, biological sequence data may represent one or more biological sequences. In certain embodiments, biological sequence data may represent a single particular type of biological sequence, such as a nucleotide sequence (e.g., DNA), a ribonucleotide sequence (e.g., RNA), and an amino acid sequence (e.g., a polypeptide, such as a protein and/or peptide, sequence). In certain embodiments, may represent multiple different types of biological sequences.

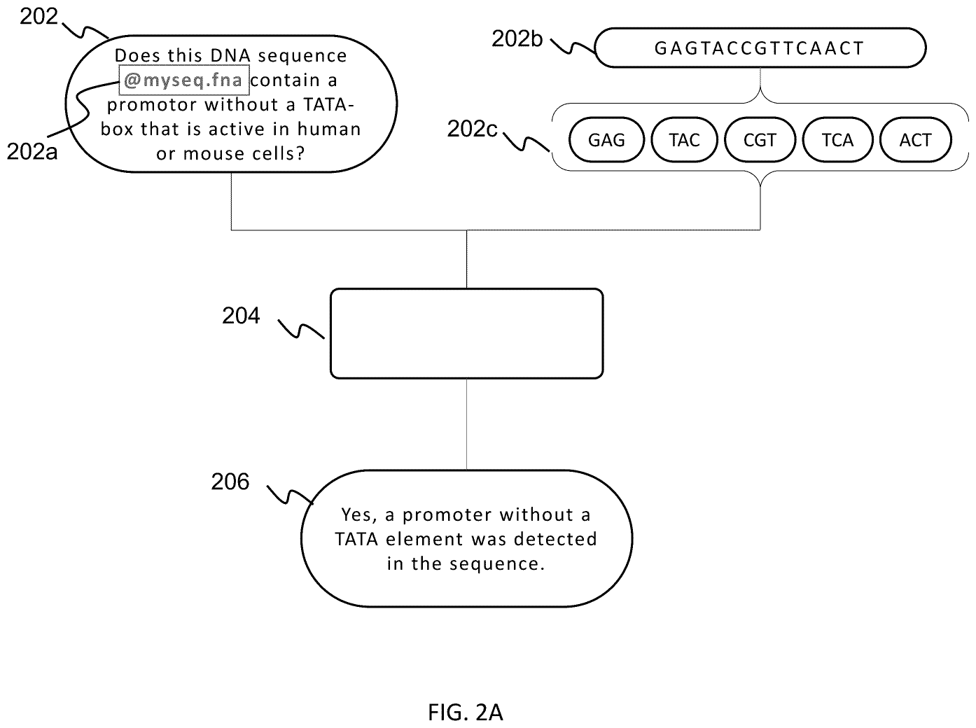

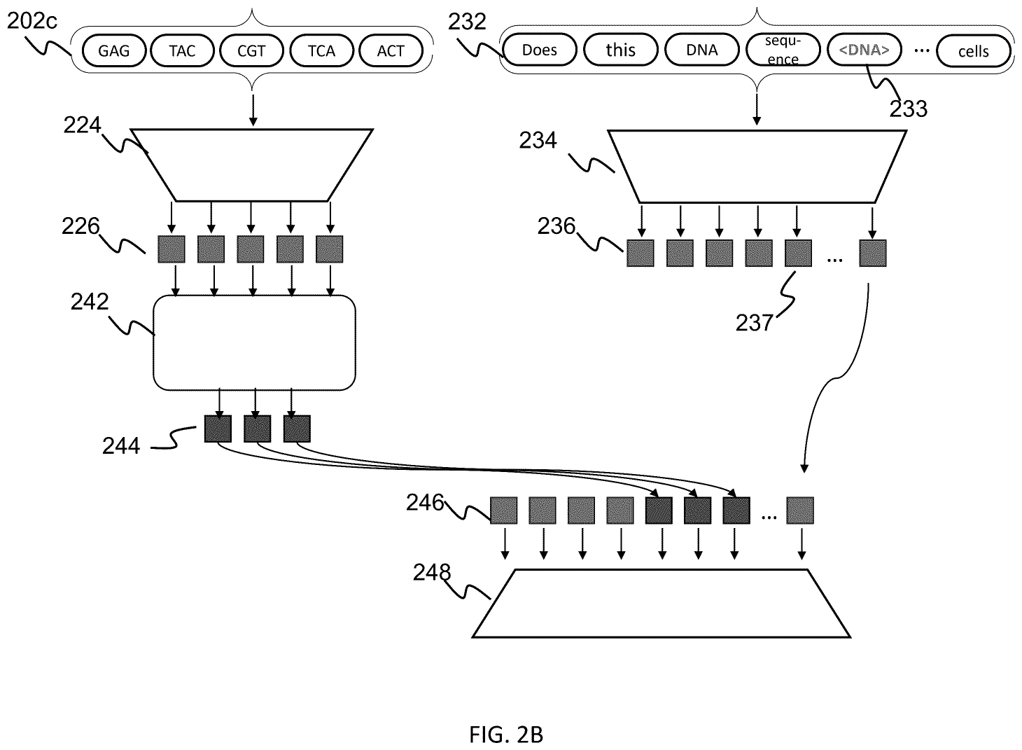

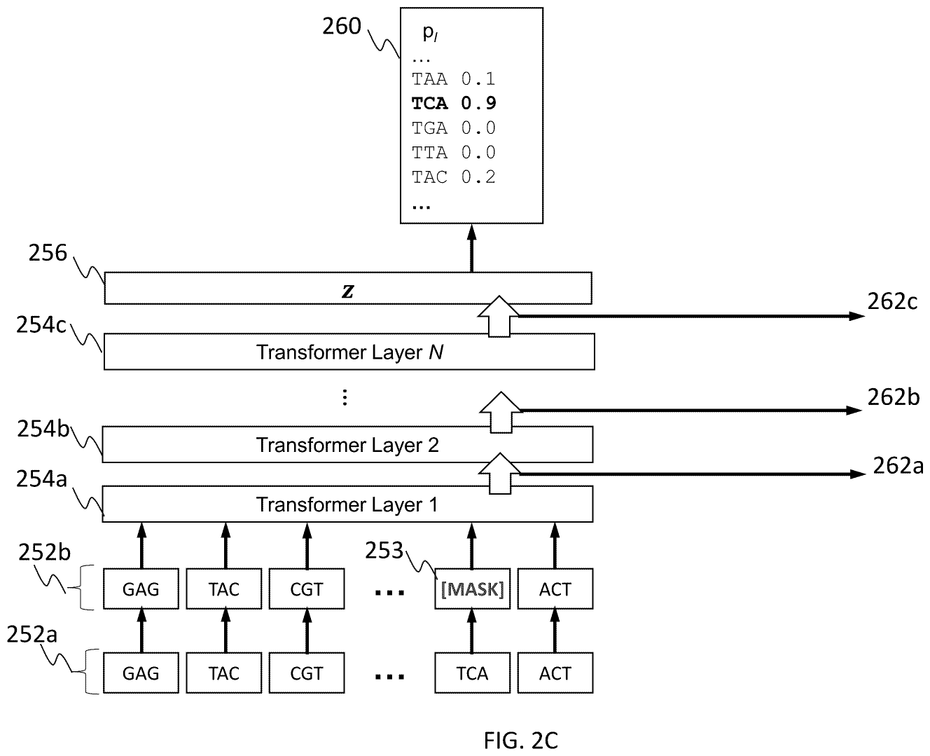

Turning to FIG. 2A, in certain embodiments, natural language prompt 202 includes a sequence tag 202a. Sequence tag 202a may encode a reference to a particular biological sequence dataset 202c (e.g., representing a single, particular biological sequence), such as via a dedicated reference format, a filename and location, a uniform resource locator (URL), a web-link, and the like. In certain embodiments, this approach allow input queries to be constructed in an efficient and convenient fashion, interleaving natural language and biological sequences. In certain embodiments, sequence tag 202a may be a positional tag, such that its particular position within natural language prompt 202 can be determined via machine learning model 204 and, accordingly, leveraged for context. In certain embodiments, multiple sequence tags may be included in a natural language prompt 202, for example allowing a single input query to include biological sequence data representing multiple biological sequences.

As shown in FIG. 2A, in certain embodiments, natural language prompt 202 together with biological sequence data 202b and/or, a tokenized version thereof 202c is provided as input to, and analyzed by, a machine learning model 204. As described in further detail herein, machine learning model 204 may comprise multiple components or sub-models, allowing it to evaluate multiple modalities, including natural language prompts 202, and generate, as output, a natural language response 206.

### B. Biological Language Models and Encoders

Turning to FIG. 2B, in certain embodiments, machine learning model 204 comprises a biological language encoder model 222 and a natural language decoder model 224.

In certain embodiments, biological language encoder 222 is used to generate 104 one or more biological sequence embeddings 226 based on biological sequence data 202b.

Biological sequence data 202b may be or comprise a computer representation of one or more biological sequences, such as one or more DNA sequences or portions thereof, and may utilize a variety of formats. Biological sequences may be represented, for example, via a sequence of alphanumeric characters, such as a sequence of letters, each representing a particular sub-unit or building block of, e.g., a particular biopolymer defined by a given biological sequence. For example, a DNA sequence may be represented as a text string with the characters “A”, “C”, “G”, and “T” representing the four naturally occurring nucleotides, adenine, cytosine, guanine, and thymine, respectively. Other manners of representing DNA sequences are also possible. For example, rather than use alphabetical characters, the numbers 1, 2, 3, and 4 may each be assigned to represent a particular naturally occurring base, and a numerical string used to represent a DNA sequence. In certain embodiments, a one-hot encoding approach is used, where each position in a DNA sequence is represented via a four-element vector, populated with zeros and a single one (1) (i.e., a one-hot vector) at a position identifying a particular nucleotide, for example as shown below.

Example one hot-encoding representation of a four-letter DNA sequence alphabet:
Adenine (A): [1 0 0 0]
Cytosine (C): [0 1 0 0]
Guanine (G): [0 0 1 0]
Thymine (T): [0 0 0 1]

In certain embodiments, biological sequence data 202b may be, or be used to generate, a tokenized representation 202c, whereby each non-overlapping set of one or more consecutive sub-units or building blocks—e.g., a k-mer (where k is an integer greater than or equal to one)—is represented by a particular token. For example, in the context of DNA sequences, non-overlapping sets of one or more nucleotides—k-mers—may be represented by tokens. In certain embodiments, sets of three, four, five, six, etc. of nucleotides are represented by a token.

For example, as shown in FIG. 2A, biological sequence 202b is a DNA sequence and may be partitioned into non-overlapping sets of three consecutive nucleic acids, such that a length L sequence is transformed to a tokenized sequence 202c of length L/3 and, instead of a four-letter alphabet, 4×4×4=64 distinct tokens are available to represent each unique three-nucleic-acid combination.

Turning again to FIG. 2B, biological language encoder 224 may receive, as input, a tokenized representation 202c of biological sequence data 202b. Said another way, biological language encoder 224 may receive, as input, a sequence of tokens 202c representing a biological sequence.

In certain embodiments, based on a sequence tokens 202c received as input, biological language encoder 224 generates one or more (e.g., a plurality of) sets of biological sequence embedding vectors (e.g., biological sequence embedding(s)).

Turning to FIG. 2C, in certain embodiments, embeddings are sets of numerical vectors that represent sequence data, which may be formatted as a sequence of tokens. Accordingly, biological sequences, as well as sequences of letters, words, etc.—i.e., natural language—can be represented via embedding vectors.

In certain embodiments, embeddings are generated using machine learning models, such as language models (LMs). In certain embodiments, an LM is or comprises one or more recurrent models, such long short-term memories (LSTMs), implemented alone or in combination, e.g., as in a bi-directional LSTM (bi-LSTM). In certain embodiments, an LM comprises one or more transformer models. Examples of LMs include, without limitation, evolutionary scale models (ESM), bidirectional encoder representations from transformers (BERT), and the like. In certain embodiments, LMs may comprise one or more members selected from the group consisting of an autoregressive LM, autoencoding LM, encoder-decoder LM, bidirectional LM, fine-tuned LMs, and multimodal LMs.

In certain embodiments, LMs are used to generate predictions about textual data representing, for example, a language. Languages processed by LMs include, without limitation, natural languages such as English, French, Thai, and the like. LMs may also operate on biological sequences, treating them as languages. LMs may, accordingly, in certain embodiments, be used to analyze and generate predictions about biological sequences, such as genetic sequences, polypeptide sequences, and the like.

For example, LMs may be used to evaluate protein sequences, treating proteins as sentences and amino acids as words. In certain embodiments, machine learning models of the present disclosure utilize an LM approach in the context of genetic sequences, treating sequences of nucleotides as sentences and tokens representing k-mers (e.g., sets of k consecutive nucleotides) as words—e.g., a “genomic” LM.

In certain embodiments, for example as illustrated in FIG. 2C, a biological LM, such as a genomic LM, may be trained to receive, as input, biological sequence data in which a biological sequence is represented via a sequence of tokens and predict values of unseen or masked tokens.

In certain embodiments, during training, a biological LM may be presented with example biological sequences in which a fraction of input tokens are masked, or with example sequences that are incomplete. For example, a central token of a sequence may be masked. For example, randomly 15% (e.g., 5%, 10%, 20%) of tokens within a sequence may be masked. A biological LM may then be tasked with predicting values of the masked tokens or next tokens in incomplete sequences. In this way, LMs can be trained in an unsupervised fashion, on unlabeled genetic sequence data.

For example, as illustrated in FIG. 2C, a LM may comprise one or more (e.g., a plurality of) transformer layers 254a, 254b, 254c and an output head 256 that outputs a set of likelihood values 260 representing predicted likelihoods of various possible types (or values) of masked token 253. By comparing this output with ground truth values, available in the original sequence, a LM can be trained to generate accurate predictions.

In certain embodiments, LMs generate, for example internally, high-dimensional representations of biological sequences, referred to as embeddings. For example, for a given sequence received as input, an embedding may comprise, for each nucleotide or token of the given sequence, a vector having a plurality of values (e.g., numerical values). That is, given a nucleotide sequence, provided as a sequence of tokens to a genomic LM, the genomic LM may generate, for each token a corresponding N-dimensional embedding vector, N is an integer, corresponding to the dimension of the embedding.

As shown in FIG. 2C, these embeddings may be extracted, and used in and of themselves as input to one or more downstream models, for example as described in further detail herein. In certain embodiments, a set of embedding vectors is extracted from a particular layer of LM. In certain embodiments, an embedding 262c is extracted from a final transformer layer 254c. In certain embodiments, a set of embedding vectors is extracted from earlier transformer layers, as illustrated in FIG. 2C. In certain embodiments, multiple sets of embedding vectors are extracted and used as embeddings, e.g., each set of embedding vectors from a particular transformer layer.

In certain embodiments, for example since sequence lengths can vary, a set of embedding vectors may be summed, averaged, or otherwise aggregated across sequence positions, for example, as described in in PCT publications WO 2022/235847 and WO 2022/235853, the content of which is hereby incorporated by reference in its entirety.

As described, for example in H. Dalla-Torre et al. 2023, the content of which is incorporated by reference herein in its entirety, embeddings generated by LMs trained on large amounts of genetic data encode valuable information about genetic sequences. Since genomic LMs can be trained in an unsupervised (or self-supervised) fashion via masked token or next token prediction approaches, they are able to leverage vast amounts of training data, which may include various genetic sequences available via public and/or proprietary sources such as the Human Genome Project (e.g., https://www.ncbi.nlm.nih.gov/datasets/genome/GCF_000001405.26/), the 1000 Genomes project, as well as various other datasets. As described in further detail herein, and demonstrated in Example 1 below, in certain embodiments, embedding vectors generated via a genomic LM can be leveraged and used as input to downstream models, such as a segmentation head, to offer improved performance on particular tasks.

In certain embodiments, a set of embedding vectors is extracted from one or more particular layers of a LM. For example, in certain embodiments, a LM may comprise a plurality of layers, and a set of embedding vectors may be extracted from a portion (e.g., not necessarily all) of the layers. For example, a single embedding vector may be extracted from a single layer, such as a final layer. In certain embodiments, a set of embedding vectors may be extracted from a plurality of layers [e.g., each embedding vector of the set corresponding to and extracted from a (e.g., different) particular layer]. In certain embodiments, a particular set of embedding layers and/or a particular set of LM layers from which embedding vectors are extracted may be determined via a probing approach. Probing may be used to assess a quality (e.g., performance) of a set of embedding vectors. For example, a LM may be trained, e.g., initially (e.g., pre-trained; e.g., to serve as a foundational model) on a first particular task, such as masked language modelling. In certain embodiments, one or more (e.g., each) layer of the LM may be probed to evaluate performance on several particular tasks to evaluate the representation capabilities of the LM. For example, given a dataset of nucleotide sequences for a task, embedding vectors returned by one or more (e.g., five, ten, twenty) layers of LM may be computed and stored. The embedding vectors of each individual layer of LM may be used as inputs for several downstream models, such as logistic regression model and multi-layer perceptron, to solve a task. A search over hyperparameters may be used for training the resulting models. For example, a model may be trained and validated using various hyperparameters, such as a learning rate, an activation function and a number of layers, to find a best performing model for a given layer of LM. Such hyperparameter search may be used to determine a best performing model associated with a specific layer of LM. The best performing models for various layers of LM may be further evaluated on a testing set. Model performances may be related with roles of associated LM layers on task performance of the LM model. The obtained insights may be used to further revise and optimize LM and its architecture to enhance its performance on tasks.

In certain embodiments, a machine learning model is (e.g., at least partially) trained while keeping some weights constant (e.g., frozen) (e.g., weights associated with one or more segmentation heads, weights associated with one or more encoders). Such training may result in improved results (e.g., faster training) in scenarios when, for example, weights associated with a particular part of a machine learning model are predominantly changing during the training as compared to other weights in the machine learning model.

In certain embodiments, at least a portion of a machine learning model is fine-tuned (e.g., using IA3 technique, e.g., from H. Liu et al. “Few-shot parameter-efficient fine-tuning is better and cheaper than in-context learning,” 2022). Specifically, at least a part of a pre-trained machine learning model (e.g., an encoder, a segmentation head) may be modified and further trained (e.g., in a supervised fashion) (e.g., separately from the remaining part of the machine learning model). For example, a segmentation head may be replaced by a classification or regression head. The segmentation or regression head may be further trained, for example, separately from the rest of the machine learning model. For example, weights of encoder layers may be kept constant (e.g., frozen) during training of a segmentation or regression head. For example, weights of encoder layers may be kept constant (e.g., frozen) and new, learnable weights are introduced. For example, for each transformer layer, three vectors with learnable weights may be introduced. The resulting model may be further trained for tasks. As transformer weights are kept frozen, new introduced weights may “fine-tune” a model to a given task, achieving greater predictive ability. Overall, fine-tuning a part of a machine learning model may lead to performance improvements.

### C. Combining Biological Sequence Embeddings with Natural Language Text Representations

Biological sequence embeddings may be combined with text-based representations of a natural language prompt for analysis and generation of response text by a natural language decoder model.

Turning again to FIG. 2B, in certain embodiments, similar to how biological sequence embeddings are generated using a biological language encoder, text embeddings may be generated based on natural language prompt using a natural language encoder. Natural prompt may be tokenized and represented as a sequence of tokens 232. A variety of tokenization approaches are feasible and may be used in the context of natural language processing. For example, approaches such as character-level tokenization, word-level tokenization, subword tokenization, sentence tokenization, and the like may be used to represent blocks of natural language text as a sequence of tokens. Various algorithms and implementations are available, including, for example, via NLTK (https://www.nltk.org/), spaCy (https://spacy.io/), Keras (https://keras.io/), LLaMA (https://huggingface.co/docs/transformers/en/model_doc/llama), etc.

In certain embodiments, text embeddings 236 are generated from a natural language prompt, for example by providing a tokenized version 232 of the natural language prompt as input to a natural language encoder 234.

In this manner, natural language prompt and biological sequence data may be used to generate corresponding sets of natural language embeddings 236 and biological sequence embeddings 226, respectively, for example via respective encoders.

In certain embodiments, biological sequence embeddings 226 and text embeddings 236 have different dimensions and, accordingly, a projection layer 242 is used to project biological sequence embeddings into a same dimension as text embeddings—i.e., generating a one or more projected embeddings 244. In certain embodiments, projection layer 242 may also resample biological sequence embeddings, such that, for example, K projected embedding vectors 244 are generated from N original biological sequence embedding vectors (K and N being integers). In certain embodiments, projection layer resamples biological sequence embedding vectors 226 such that a fixed, fewer number of projected embedding 244 vectors are generated for a given input set of biological sequence embedding vectors.

In certain embodiments, e.g., as shown in FIG. 2B, projection layer 242 receives biological sequence embedding vectors as input. In certain embodiments, for example as shown and described in Example 1, below, a natural language-aware projection layer may (e.g., in addition to biological sequence embeddings) receive text embeddings as input. In this way, projection layer may, among other things, leverage context and/or metadata provided via natural language prompt and text embeddings 236 determined therefrom to create improved projected embeddings whose values depend not only on the particular biological sequence data received as input, but, e.g., additionally, on the context in which it is referred to via in the received natural language prompt and, additionally or alternatively, any additional related information—e.g., metadata—describing it (e.g., a particular type of organism, cell, etc.).

In certain embodiments, projected embedding vectors 244 are inserted into a sequence of text embedding vectors. For example, a natural language prompt with a positional sequence tag may represent positional sequence tag via a placeholder token 233, which, in turn, allows a position 237 in a sequence of embedding vectors to be identified. Projected embedding vectors 244 are then inserted in place of sequence placeholder at the appropriate location 237 in the sequence of text embedding vectors 236, thereby generating a combined embedding 246. Accordingly, as shown in FIG. 2B, combined embedding 246 comprises a set of text embedding vectors interleaved with projected embedding vectors derived from biological sequence data. Combined embedding 246 may be used as input to a natural language decoder 248, to generate a response as output. In this manner, biological sequence data and natural language prompts are harmonized and can be processed in a unified fashion.

Among other things, not only does this approach provide a convenient and user friendly conversational style interface and output format for interfacing with powerful underlying machine learning techniques, but, moreover, as described in the following section, the approach offers improvements in training efficiency, facilitates multi-task training, and provides a framework that can readily be extended to other biological modalities.

### D. Training Multi-Modal Composite Models

As shown in FIG. 2B, multi-modal machine learning models that harmonize natural language prompts with biological sequence data comprise multiple sub-models, including, e.g., biological sequence encoder, projection layer, and natural language encoder and/or decoders.

In certain embodiments, as described herein, biological sequence encoder and natural language encoder and/or decoders are pre-trained models. These models may be trained separately, in an unsupervised fashion, for example, as described herein with regard to FIG. 2C.

In certain embodiments, projection layer 242 may be trained using a question-and-answer dataset comprising example natural language prompts and corresponding target answers. In certain embodiments, each example prompt may be a textualized version of a particular task, including various classification and/or regression tasks, e.g., as described in Example 1, below. An architecture such as the one shown in FIGS. 2A and 2B, comprising projection layer 242 along with biological sequence encoder 224 and natural language encoder 234 and decoder 248 may be used to generate, for each example prompt, a corresponding determined response. During training, determined responses may be compared with target answers and a training loss computed and used to update weights of various learnable parameters of projection layer 242.

In certain embodiments, projection layer 242 is trained in this manner, while biological sequence encoder 224, natural language encoder 234 and natural language decoder 248 are frozen (i.e., values of their learnable parameters held fixed), having been previously pre-trained, for example in an unsupervised fashion as described herein. In certain embodiments, while biological sequence encoder 224 may initially be pre-trained, it may, subsequently, be further tuned by allowing its parameter values to vary along with those of projection layer 242 during the supervised question and answer training procedure described above.

Among other things, beyond allowing machine learning model 204 to receive instructions and generate responses in a convenience, user-friendly, natural language format, by allowing all input and output to be cast in a single, unified, format, training can be performed in a fashion that is agnostic to the particular task itself. In this way, technologies of the present disclosure may be trained to minimize a unified objective for all tasks, which may, for example as described in Example 1, take the form of a cross-entropy loss between machine-learning model-determined response and the target answers (e.g., tokens). This single objective allows training to proceed seamlessly across tasks without introducing conflicting gradients or scale issues that might ordinarily arise due to different objectives and loss functions associated with different tasks. Not only does this facilitate training, but it is believed to improve model performance, since training on multiple related tasks allows for transfer learning, whereby information (e.g., encoded in adjustments to learnable parameter values) determined via training steps on examples pertaining to one task may complement information determined via training on other tasks.

### E. Example Combined Nucleotide Sequence and Natural Language Model and Extensions to other Modalities

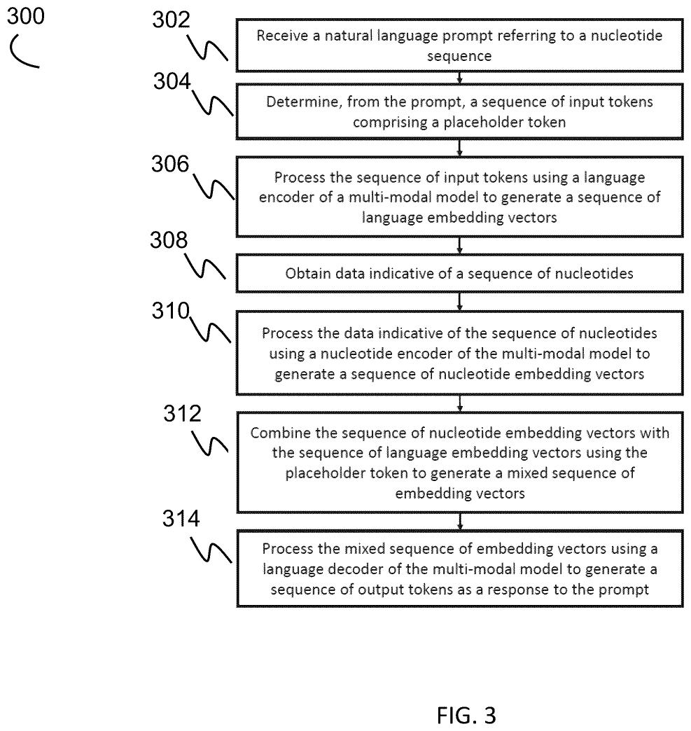

Among other things, as illustrated in FIG. 3 and demonstrated in detail in Example 1, below, approaches described above may be used in the context of a multi-modal model that allows for natural language prompts to provide instructions for biological sequence [e.g., nucleotide sequence (e.g., DNA sequence)] processing tasks.

For example, in example process 300, a natural language prompt referring to a biological sequence may be received 302. As described herein, natural language prompt may use a positional sequence tag to refer to a biological sequence and may determine 304, from the prompt, a sequence of input tokens comprising a placeholder token (e.g., corresponding to positional sequence tag). The sequence of input tokens may be processed 306 using a language encoder to generate a sequence of language (e.g., text) embedding vectors.

In certain embodiments, biological sequence data representing the nucleotide sequence is obtained 308 and processed using a nucleotide encoder to generate a sequence of nucleotide embedding vectors 310. Biological sequence embedding vectors 310 and language embedding vectors may, accordingly, be combined as described herein to generate a mixed sequence of embedding vectors 312. The mixed sequence may be processed using a language decoder to generate 314 a sequence of output tokens representing a response to the natural language prompt received as input.

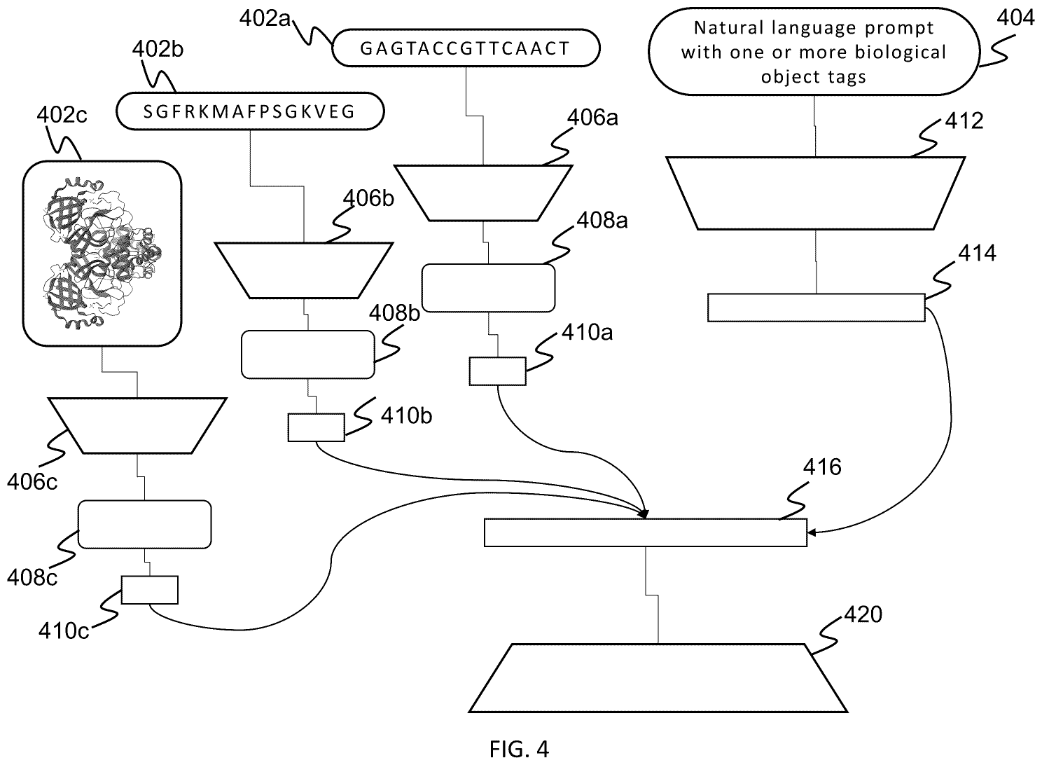

Turning to FIG. 4, additionally or alternatively, in certain embodiments technologies of the present disclosure may accommodate multiple types of biological object data in connection with the natural language prompt-response format described herein. Biological object data may include multiple types of biological sequences, such as nucleotide sequences 402a and polypeptide sequences 402b. Biological object data may also include non-sequence data, such as structural data 402c representing a 3D structure of a biological molecule, such as 3D DNA, RNA, and/or protein structure. Natural language prompt 404 may include various biological object tags, each identifying a particular biological object and its corresponding data.

As shown in FIG. 4, in certain embodiments, each particular type of biological object data may be associated with a particular encoder (e.g., and projection layer), having been trained to generate a set of embedding vectors for that particular type of biological object data. For example, as illustrated in FIG. 4, nucleotide sequence data may be associated with, and processed by, a corresponding nucleotide language encoder 406a and projection layer 408a, to generate a set of projected nucleotide sequence embeddings 410a. Polypeptide (e.g., protein) sequence data 402b may be associated with, and processed by, a corresponding protein language encoder 406b and projection layer 408b, to generate a set of protein sequence embeddings 410b. Structural data 402c may be associated with, and processed by, a corresponding structural data encoder 406c and projection layer 408c, to generate a corresponding set of structural data embedding vectors 410c.

In certain embodiments, a single, multi-omic, language encoder may be trained and used to process multiple distinct types of sequences, such as DNA and RNA sequences, DNA and/or RNA and protein sequences, etc.

Natural language prompt may be processed by a natural language encoder 412 and a correspond set of text embedding vectors 414 generated. In certain embodiments, guided by one or more biological object tags present in natural language prompt, projected embedding vectors representing biological object data of various formats, such as nucleotide sequence data, protein sequence data, structural data, etc., may be combined with text embeddings 414 to generate a set of mixed embedding vector 416. These mixed embeddings 416 may then, as described herein, be passed to a natural language decoder 420 to generate, e.g., as output, a text response (e.g., a set of tokens representing a natural language response).

Accordingly, among other things, methods and systems of the present disclosure provide a framework that can be adapted to accommodate a variety of datatypes together with a natural language input/output format.

### F. Software, Computer System, and Network Environment

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), random forest, decision trees, support vector machines, and the like, in order to determine, for a given input, one or more output values. In certain embodiments, the input comprises alphanumeric data which can include numbers, words, phrases, or lengthier strings, for example. In certain embodiments, the one or more output values comprise values representing numeric values, words, phrases, or other alphanumeric strings. In certain embodiments, the one or more output values comprise an identification of one or more response strings (e.g., selected from a database).

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task such as identifying certain response strings, values of determined parameters are fixed and the (e.g., unchanging, static) machine learning module is used to process new data (e.g., different from the training data; e.g., infer a result) and accomplish its trained task without further updates to its parameters (e.g., the machine learning module does not receive feedback and/or updates). In certain embodiments, machine learning modules may receive feedback, e.g., based on automated review of accuracy or human user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC), field programmable gate arrays (FPGAs)).

In certain embodiments, machine learning modules implementing machine learning techniques may be composed of individual nodes (e.g., units, neurons). A node may receive a set of inputs that may include at least a portion of a given input data for the machine learning module and/or at least one output of another node. A node may have at least one parameter to apply and/or a set of instructions to perform (e.g., mathematical functions to execute) over the set of inputs. In certain embodiments, node instructions may include a step to provide various relative importance to the set of inputs using various parameters, such as weights. The weights may be applied by performing scalar multiplication (e.g., or other mathematical function) between a set of inputs values and the parameters, resulting in a set of weighted inputs. In certain embodiments, a node may have a transfer function to combine the set of weighted inputs into one output value. A transfer function may be implemented by a summation of all the weighted inputs and the addition of an offset (e.g., bias) value. In certain embodiments, a node may have an activation function to introduce non-linearity into the output value. Nonlimiting examples of the activation function include Rectified Linear Activation (ReLu), logistic (e.g., sigmoid), hyperbolic tangent (tanh), and softmax. In certain embodiments, a node may have a capability of remembering previous states (e.g., recurrent nodes). Previous states may be applied to the input and output values using a set of learning parameters.

A layer is a building block in a deep learning architecture composed of nodes. A layer is a set of nodes that receives data input (e.g., weighted or non-weighted input), transforms it (e.g., by carrying out instructions, e.g., applying a set of functions e.g., linear and/or non-linear functions), and passes transformed values as output (e.g., to the next layer). In certain embodiments, the set of nodes in a particular layer may share the same parameters and instructions without interacting with each other. A machine learning module may be composed of at least one layer (e.g., ordered). Examples of types of layers include convolutional layers (e.g., layers with a kernel, a matrix of parameters that is slid across an input to be multiplied with multiple input values to reduce them to a single output value); fully connected (FC) layers (e.g. all nodes are connected to all outputs of the previous layer); recurrent layers, long/short term memory (LSTM) layers, gated recurrent unit (GRU) layers (e.g., nodes with the various abilities to memorize and apply their previous inputs and/or outputs); batch normalization (BN) layers (e.g., layers that normalize a set of outputs from another layer, allowing for more independent learning of individual layers); activation layer (e.g., layers with nodes that only contain an activation function); (un)pooling layers [e.g., layers that reduce (increase) dimensions of an input by summarizing (splitting) input values in defined patches).

In certain embodiments, the performance of a machine learning module may be characterized by its ability to produce an output data that reproduces an input data with specific accuracy. To achieve specific accuracy, a training process is performed to find optimal parameters, such as weights, for every node in every layer of the machine learning module. In certain embodiments, the training process of a machine learning module may involve using output data to calculate an objective function (e.g., cost function, loss function, error function) that needs to be optimized (e.g., minimized, maximized). For example, a machine learning objective function may be a combination of a loss function and regularization parameter. The loss function is related to how well the output is able to predict the input. The loss function may take various forms, like mean squared error, mean absolute error, binary cross-entropy, categorical cross-entropy, for example. The regularization term may be needed to prevent overfitting and improve generalization of the training process. Typical regularization techniques include L1 Regularization or Lasso Regression, L2 Regularization or Ridge Regression, and Dropout (e.g., dropping layer outputs at random during training process).

In certain embodiments, objective function optimization of a machine learning module may involve finding at least one (e.g., all) of the present global optima (e.g., as opposed to local optima). A typical algorithm for objective function optimization follows principles of mathematical optimization for a multi-variable function and relies on achieving specific accuracy of the process. Examples of objective function optimization algorithms include gradient descent, nonlinear conjugate gradient, random search, Levenberg-Marquardt algorithm, limited-memory Broyden-Fietcher-Goldfarb-Shanno algorithm, pattern search, basin hopping method, Krylov method, Adam method, genetic algorithm, particle swarm optimization, surrogate optimization, and simulated annealing.

In certain embodiments, available input data includes training data and validation data, e.g., where the validation data is separate and non-overlapping with the training data. Training data is used during the training process to optimize a model, whereas validation data is used to check the accuracy of the model while operating on previously unseen data. In certain embodiments, training data is divided into batches (e.g., portions) that is sequentially used (e.g., in random order) as sets of inputs to train a model. In certain embodiments, a model is trained multiple times (e.g., epochs) on the entire set of training data.

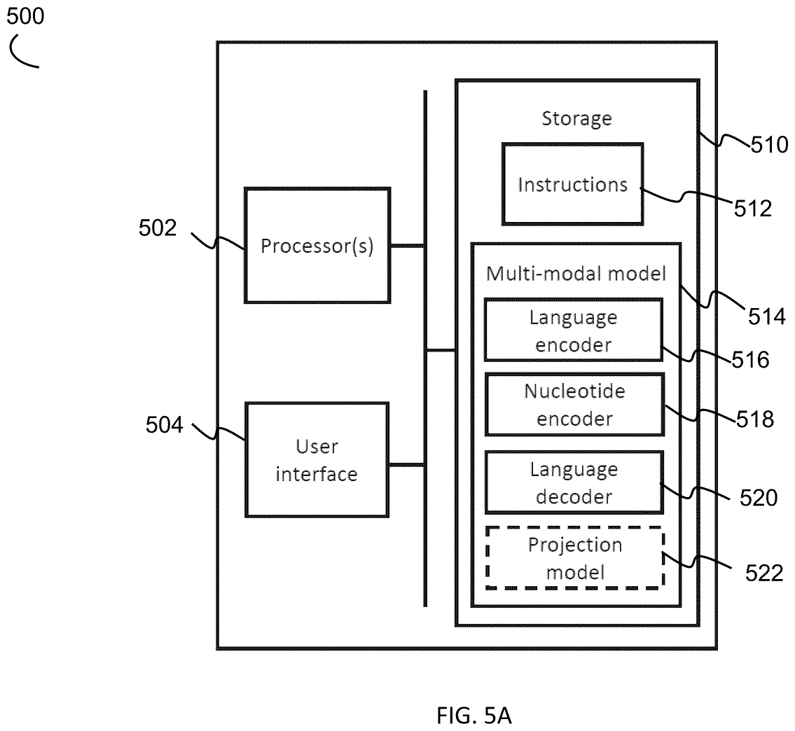

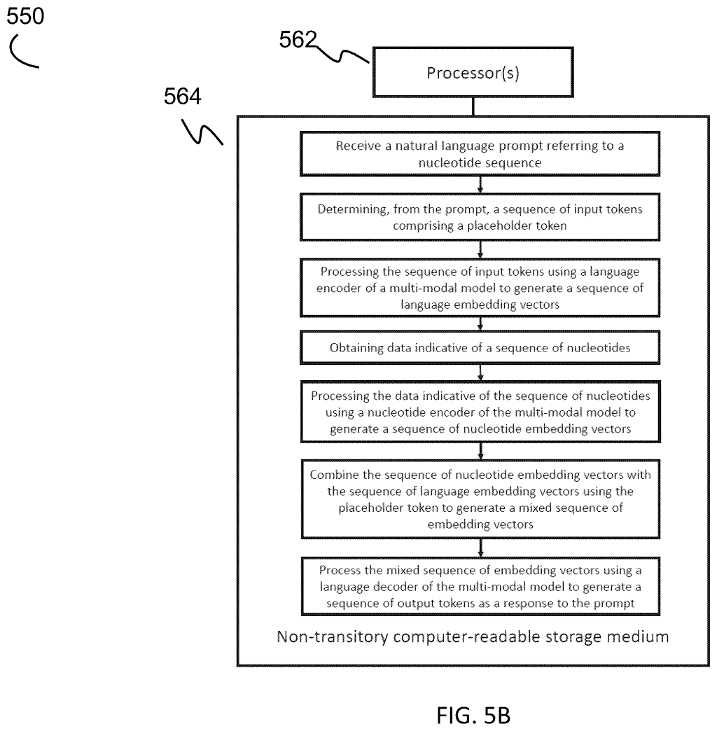

Turning to FIGS. 5A-5B, various processes and machine learning models used, e.g., in connection with processes described herein, may be included and/or stored in various computer systems and computer-readable media, in certain embodiments. FIG. 5A shows an exemplary system 500 for carrying certain methods of the present disclosure. The system 500 may comprise a processor 502, a user interface 504, and a storage medium 510 with stored instructions 512 as well as data (e.g., hyperparameters, weights) 514 associated with a machine learning model (e.g., a multi-modal model) various sub-models components, such as a natural language encoder 516, one or more biological object encoders, such as a nucleotide (sequence) encoder 518, a natural language decoder 520 and, optionally, projection model 522. FIG. 5B shows an example processor 562 in communication with a non-transitory medium 564, storing instructions for carrying out methods described herein, such as those in FIG. 3.

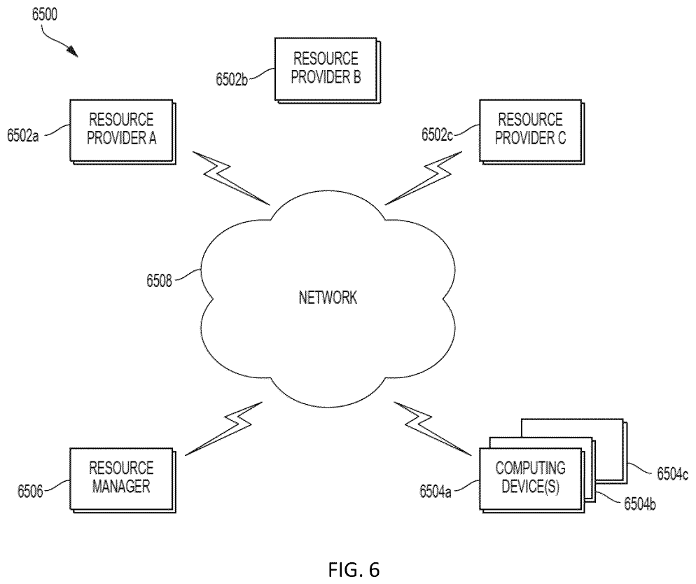

In certain embodiments, technologies of the present disclosure may be provided using a network environment. For example, as shown in FIG. 6, an implementation of a network environment 6500 for use in providing systems, methods, and architectures as described herein is shown and described. In brief overview, referring now to FIG. 6, a block diagram of an exemplary cloud computing environment 6500 is shown and described. The cloud computing environment 6500 may include one or more resource providers 6502a, 6502b, 6502c (collectively, 6502). Each resource provider 6502 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 6502 may be connected to any other resource provider 6502 in the cloud computing environment 6500. In some implementations, the resource providers 6502 may be connected over a computer network 6508. Each resource provider 6502 may be connected to one or more computing device 6504a, 6504b, 6504c (collectively, 6504), over the computer network 6508.

The cloud computing environment 6500 may include a resource manager 6506. The resource manager 6506 may be connected to the resource providers 6502 and the computing devices 6504 over the computer network 6508. In some implementations, the resource manager 6506 may facilitate the provision of computing resources by one or more resource providers 6502 to one or more computing devices 6504. The resource manager 6506 may receive a request for a computing resource from a particular computing device 6504. The resource manager 6506 may identify one or more resource providers 6502 capable of providing the computing resource requested by the computing device 6504. The resource manager 6506 may select a resource provider 6502 to provide the computing resource. The resource manager 6506 may facilitate a connection between the resource provider 6502 and a particular computing device 6504. In some implementations, the resource manager 6506 may establish a connection between a particular resource provider 6502 and a particular computing device 6504. In some implementations, the resource manager 6506 may redirect a particular computing device 6504 to a particular resource provider 6502 with the requested computing resource.

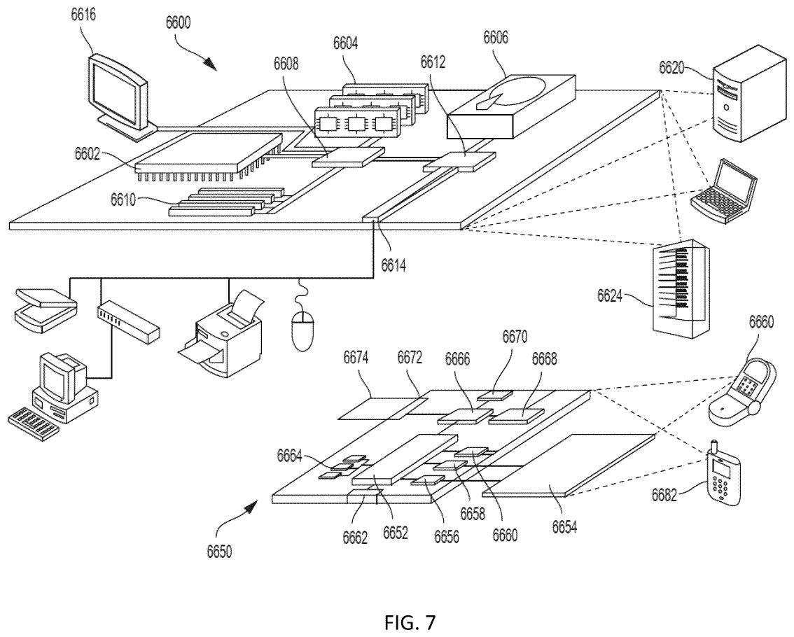

FIG. 7 shows an example of a computing device 6600 and a mobile computing device 6650 that can be used to implement the techniques described in this disclosure. The computing device 6600 is intended to represent various forms of digital computers, such as laptops, desktops, workstations, personal digital assistants, servers, blade servers, mainframes, and other appropriate computers. The mobile computing device 6650 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 6600 includes a processor 6602, a memory 6604, a storage device 6606, a high-speed interface 6608 connecting to the memory 6604 and multiple high-speed expansion ports 6610, and a low-speed interface 6612 connecting to a low-speed expansion port 6614 and the storage device 6606. Each of the processor 6602, the memory 6604, the storage device 6606, the high-speed interface 6608, the high-speed expansion ports 6610, and the low-speed interface 6612, are interconnected using various busses, and may be mounted on a common motherboard or in other manners as appropriate. The processor 6602 can process instructions for execution within the computing device 6600, including instructions stored in the memory 6604 or on the storage device 6606 to display graphical information for a GUI on an external input/output device, such as a display 6616 coupled to the high-speed interface 6608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 6604 stores information within the computing device 6600. In some implementations, the memory 6604 is a volatile memory unit or units. In some implementations, the memory 6604 is a non-volatile memory unit or units. The memory 6604 may also be another form of computer-readable medium, such as a magnetic or optical disk.

The storage device 6606 is capable of providing mass storage for the computing device 6600. In some implementations, the storage device 6606 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 6602), perform one or more methods, such as those described above. The instructions can also be stored by one or more storage devices such as computer- or machine-readable mediums (for example, the memory 6604, the storage device 6606, or memory on the processor 6602).

The high-speed interface 6608 manages bandwidth-intensive operations for the computing device 6600, while the low-speed interface 6612 manages lower bandwidth-intensive operations. Such allocation of functions is an example only. In some implementations, the high-speed interface 6608 is coupled to the memory 6604, the display 6616 (e.g., through a graphics processor or accelerator), and to the high-speed expansion ports 6610, which may accept various expansion cards (not shown). In the implementation, the low-speed interface 6612 is coupled to the storage device 6606 and the low-speed expansion port 6614. The low-speed expansion port 6614, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 6600 may be implemented in a number of different forms, as shown in the figure. For example, it may be implemented as a standard server 6620, or multiple times in a group of such servers. In addition, it may be implemented in a personal computer such as a laptop computer 6622. It may also be implemented as part of a rack server system 6624. Alternatively, components from the computing device 6600 may be combined with other components in a mobile device (not shown), such as a mobile computing device 6650. Each of such devices may contain one or more of the computing device 6600 and the mobile computing device 6650, and an entire system may be made up of multiple computing devices communicating with each other.

The mobile computing device 6650 includes a processor 6652, a memory 6664, an input/output device such as a display 6654, a communication interface 6666, and a transceiver 6668, among other components. The mobile computing device 6650 may also be provided with a storage device, such as a micro-drive or other device, to provide additional storage. Each of the processor 6652, the memory 6664, the display 6654, the communication interface 6666, and the transceiver 6668, are interconnected using various buses, and several of the components may be mounted on a common motherboard or in other manners as appropriate.

The processor 6652 can execute instructions within the mobile computing device 6650, including instructions stored in the memory 6664. The processor 6652 may be implemented as a chipset of chips that include separate and multiple analog and digital processors. The processor 6652 may provide, for example, for coordination of the other components of the mobile computing device 6650, such as control of user interfaces, applications run by the mobile computing device 6650, and wireless communication by the mobile computing device 6650.

The processor 6652 may communicate with a user through a control interface 6658 and a display interface 6656 coupled to the display 6654. The display 6654 may be, for example, a TFT (Thin-Film-Transistor Liquid Crystal Display) display or an OLED (Organic Light Emitting Diode) display, or other appropriate display technology. The display interface 6656 may comprise appropriate circuitry for driving the display 6654 to present graphical and other information to a user. The control interface 6658 may receive commands from a user and convert them for submission to the processor 6652. In addition, an external interface 6662 may provide communication with the processor 6652, so as to enable near area communication of the mobile computing device 6650 with other devices. The external interface 6662 may provide, for example, for wired communication in some implementations, or for wireless communication in other implementations, and multiple interfaces may also be used.

The memory 6664 stores information within the mobile computing device 6650. The memory 6664 can be implemented as one or more of a computer-readable medium or media, a volatile memory unit or units, or a non-volatile memory unit or units. An expansion memory 6674 may also be provided and connected to the mobile computing device 6650 through an expansion interface 6672, which may include, for example, a SIMM (Single In Line Memory Module) card interface. The expansion memory 6674 may provide extra storage space for the mobile computing device 6650, or may also store applications or other information for the mobile computing device 6650. Specifically, the expansion memory 6674 may include instructions to carry out or supplement the processes described above, and may include secure information also. Thus, for example, the expansion memory 6674 may be provide as a security module for the mobile computing device 6650, and may be programmed with instructions that permit secure use of the mobile computing device 6650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 6652), perform one or more methods, such as those described above. The instructions can also be stored by one or more storage devices, such as one or more computer- or machine-readable mediums (for example, the memory 6664, the expansion memory 6674, or memory on the processor 6652). In some implementations, the instructions can be received in a propagated signal, for example, over the transceiver 6668 or the external interface 6662.

The mobile computing device 6650 may communicate wirelessly through the communication interface 6666, which may include digital signal processing circuitry where necessary. The communication interface 6666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 6668 using a radio-frequency. In addition, short-range communication may occur, such as using a Bluetooth®, Wi-Fi™, or other such transceiver (not shown). In addition, a GPS (Global Positioning System) receiver module 6670 may provide additional navigation- and location-related wireless data to the mobile computing device 6650, which may be used as appropriate by applications running on the mobile computing device 6650.

The mobile computing device 6650 may also communicate audibly using an audio codec 6660, which may receive spoken information from a user and convert it to usable digital information. The audio codec 6660 may likewise generate audible sound for a user, such as through a speaker, e.g., in a handset of the mobile computing device 6650. Such sound may include sound from voice telephone calls, may include recorded sound (e.g., voice messages, music files, etc.) and may also include sound generated by applications operating on the mobile computing device 6650.

The mobile computing device 6650 may be implemented in a number of different forms, as shown in the figure. For example, it may be implemented as a cellular telephone 6680. It may also be implemented as part of a smart-phone 6682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

### G. Example 1: Implementation and Performance Analysis of Exemplary Multimodal Conversational Agent—ChatNT

This example describes and demonstrates performance of an exemplary multimodal conversational agent for biological sequence analysis, denoted “ChatNT,” in accordance with certain embodiments of systems and methods described herein.

Among other things, understanding how cells, tissues, and organisms interpret information encoded in their genomes is of paramount importance for advancing understanding of fundamental biological processes and their impact, e.g., on the detection and treatment of various disease. DNA sequences of organism comprise instructions that encode RNAs and/or proteins to be produced, as well as when and in which cellular context particular RNAs and/or proteins should be produced. Since the human genome was sequenced [1], a significant focus of research has been on identifying every genomic element, characterizing their function, and assessing the impact of genetic variants on the different gene regulatory and cellular processes. Given the complexity of biological sequences and processes, and the increasing volume of genomics data, several machine learning and deep learning methods have been developed to address these questions by predicting diverse molecular phenotypes [2, 3, 4]. Examples include tasks such as predicting the binding of proteins to DNA and RNA [5, 6], DNA methylation [7], chromatin features [8, 9, 10], regulatory elements [11], 3D genome folding [12, 13, 14], splicing [15, 16], gene expression [17, 18, 10], mRNA properties such as stability [19] and polyadenylation [20, 21], and protein properties such as melting point [22].

While deep learning models used in examples such as these are typically supervised models—that is, models that are trained on (often painstakingly curated) labeled datasets where each example sequence is paired with a ground truth label representing the desired output, their performance remains often limited due to the scarcity of labeled data, given the significant time and costs associated with creating labeled datasets. Although labeled data is limited, an exponentially increasing volume of raw, unlabeled, genome data is becoming available thanks to the increase in throughput and reduced cost of modem sequencing techniques. This, accordingly, creates a significant opportunity for self-supervised deep learning methods, which can be trained on this wealth of unlabeled data. Through learning-techniques such as masked- or next-token prediction [23, 24, 25], with tokens representing one or several consecutive nucleotides, deep learning models can build powerful foundation representations of the genome during this “pretraining” stage, aggregating correlations between nucleotides and larger sequence patterns into rich high-dimensional vectors that capture known genomic elements and protein binding sites [26]. These models can themselves, later, exploit these rich representations, during a “fine-tuning” stage, to learn faster and reach better performance on supervised tasks, i.e., tasks where labels are available, despite data scarcity. Recently, several such foundation models have been built in this fashion, showing that they can be pre-trained on the genomes of hundreds of species before being fine-tuned to solve a large collection of molecular phenotype prediction tasks [26, 27, 28, 29, 30, 31, 32].

This being said, the performance and application domain of current DNA foundation models remains limited. In the current paradigm, foundation models require fine-tuning to each specific task individually to produce accurate representations and predictions, and are thus better characterized as narrow experts on specific tasks. This not only yields a deluge of different models as the number of tasks increases, but also prevents any transfer between supervised tasks as well as to solve new tasks in a zero-shot setting (i.e., without the need for further finetuning on some examples). There is therefore a need to rethink the development of genomics Artificial Intelligence (AI) systems with the goal of establishing general, unified models that capture the intricate relationships between all diverse biological sequences and functions. It has been shown in other fields such as natural language processing (NLP) and computer vision that training on several tasks in parallel results in knowledge transfer between tasks and improved accuracy and generalization [23, 24, 33, 34, 35]. In these domains, English language also plays a wider role: a universal interface for representing various tasks and instructions and helping guide the training of end-to-end multi-task models [36, 37]. Transferring this type of approaches to biological data is a promising approach towards developing a general model that can solve all genomics tasks of interest simultaneously and with improved accuracy.

An additional important aspect of building a universal genomics AI system is its accessibility to different types of users. Most biologists do not know how to use current genomics models, let alone how to program one themselves for a given task of interest. Such models are not conversational and thus of limited utility in practice to users with no coding capabilities. Also here, language can play an important role as a universal interface for a general-purpose AI assistant that can solve genomics tasks through task instructions that can be explicitly represented in English language. For example, the recent success of ChatGPT [38] and GPT-4 [39] has demonstrated the power of large language models (LLMs) trained to follow human instructions, and how such tools can transform several industries due to their ease of use. The technologies described in this present example may allow for a similar paradigm shift for genomics and biology by providing for conversational agents that are proficient in biological tasks.

To that end, this example introduces an approach to build foundation models for genomics. Drawing inspiration from NLP and recent vision/language multimodal models [24, 25 36, 40, 41, 42, 43, 44, 45, 46], to the approaches described in this example formulate all supervised genomics prediction tasks as text-to-text tasks and to build a multi-modal DNA/language agent, dubbed the “Chat Nucleotide Transformer” (or “ChatNT”). ChatNT can be given one or several DNA sequences and is prompted in English to solve all those tasks. This formulation allows all tasks to be expressed with the same vocabulary, being here the concatenation of the English and DNA vocabularies. Among other things, machine learning models can, accordingly, be trained to solve a diverse set of tasks by minimizing a unified objective [25, 47], allowing for seamless new task integration and generalization. Formulating tasks in English also provides a way to provide additional meta-data information to the model, such as the species, the chromosome or the cell type, that is also missing in most current DNA foundation models.

ChatNT is built to act as a generalist genomics AI system—a unified model that can interpret multiple biological sequences and handle dozens of tasks in a conversational agent setting. To the best of the inventors' knowledge, ChatNT is the first multimodal bio-sequence/English agent. Moreover, this example includes creation of the first datasets of genomics instructions tasks with curated sets of questions and instructions in English was created for diverse classification and regression tasks.

As demonstrated herein, ChatNT achieves a new state-of-the-art performance on the Nucleotide Transformer benchmark [26]. ChatNT was further evaluated in additional biologically relevant tasks that cover DNA, RNA and protein processes. ChatNT achieves state-of-the-art performance across all tasks, matching the performance of several specialized models, such as APARENT2 for RNA polyadenylation [20, 21] and ESM2 for protein-related tasks [48], while being able to solve a large collection of tasks at once and in English. Finally, its English conversational capabilities make its use easier than other models, widening its accessibility to scientists with no machine learning or computer science background. This framework for genomics instruction-tuning can be easily extended to new tasks or biological data modalities (e.g., sequencing experiments, imaging) without the need for pre-training from scratch every time, making it a widely applicable tool for biology.

G.i. ChatNT: A Unified Framework to Transform DNA Foundation Models into Conversational Agents to Solve Multiple Tasks

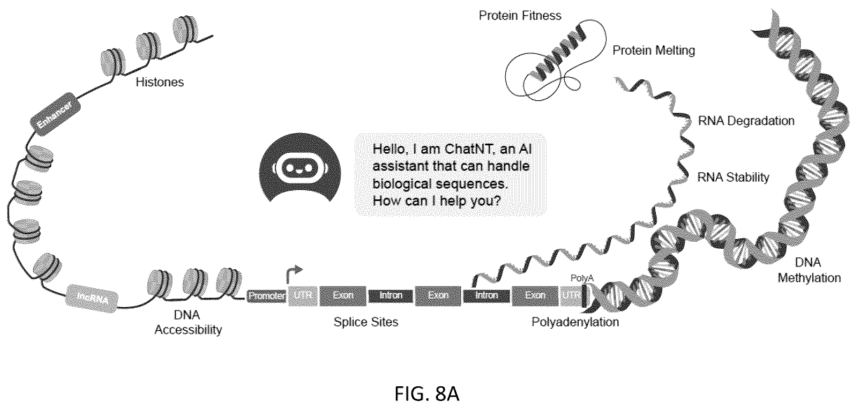

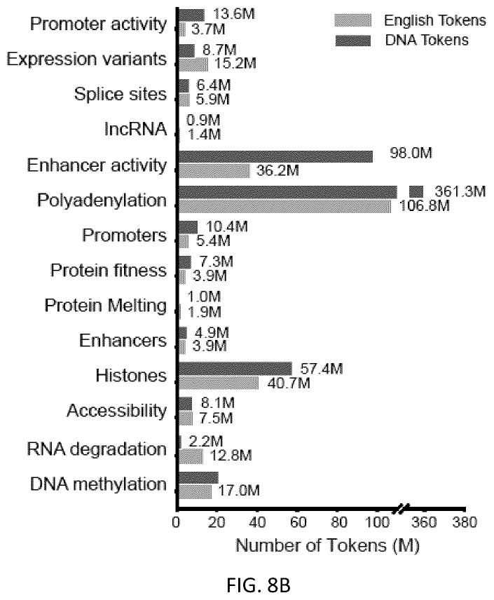

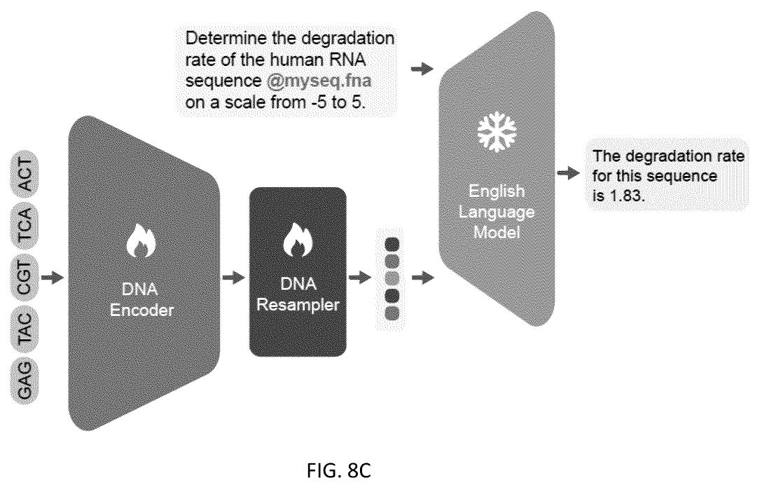

ChatNT is the first framework (to the best of knowledge of inventors) for genomics instruction-tuning, extending instruction-tuning agents to the multimodal space of biology and biological sequences as shown in FIG. 8A. FIG. 8B shows ChatNT relies on a database with a vast number of both English and DNA tokens for various tasks. The framework is designed to be modular and trainable end-to-end. The framework combines (1) a DNA encoder model, pre-trained on raw genome sequencing data and that provides DNA sequence representations; (2) an English decoder, typically a pre-trained GPT-style LLM, to comprehend the user instructions and produce responses; and (3) a projection layer that projects the representations extracted by the DNA encoder into the embedding space of the input English words, such that both can be used by the English decoder as shown in FIG. 8C. In contrast to most multimodal works (e.g. [40, 49]) that would typically freeze the encoder and train only the projection, and sometimes the decoder, it was decided herein to backpropagate the gradients in the encoder in addition to the projection to allow supervised knowledge propagation at the DNA model level. The English decoder is kept frozen and therefore ChatNT benefits from its entire initial conversational capabilities, ensuring these do not degrade during training. The Nucleotide Transformer v2 (500M) model is used for the DNA encoder part [26] and Vicuna-7b (instruction-fine-tuned LLaMA model with 7B parameters) for the English decoder part [50] in order to build the conversational agent ChatNT. Keeping this modular architecture allows for use of constantly improving encoders and decoders in the future without changing the model architecture.

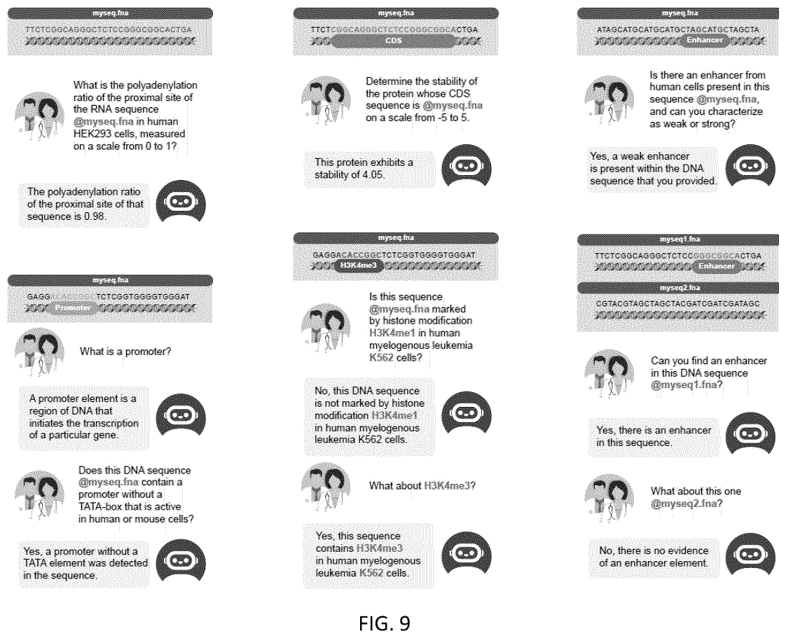

To train and evaluate ChatNT, datasets of genomics tasks were converted into instructions datasets by framing each task in English as shown in FIG. 9. For every task, a train and test file were created, each containing the respective DNA sequences combined with curated questions and answers in English. FIG. 8C shows an example of question and answer for predicting RNA degradation levels: “User: Determine the degradation rate of the human RNA sequence @myseq.fna on a scale from −5 to 5. ChatNT: The degradation rate for this sequence is 1.83.”, where the projected embeddings of the candidate DNA sequence are inserted at the @myseq.fna position. The same train/test splits are kept as the original sources of each task, and different questions are used for train and test to assess the English generalization capabilities of the model. This allows to not only evaluate the agent capability to generalize between DNA sequences but also its robustness to the English language used. A flexible way is also provided to interleave English and DNA sequences through the usage of positional tags (@myseq.fna), allowing users to refer to several sequences in the same question.

ChatNT is trained to solve all tasks simultaneously, with a uniform sampling over tasks per batch. Multi-tasking is achieved by ChatNT by prompting in natural language, where the question asked by the user will guide the agent towards the task of interest. Given a text prompt and one or multiple DNA sequences as input, ChatNT is trained to minimize a unified objective for all tasks, which takes the form of the cross-entropy loss between ChatNT predictions and the target answer tokens, as in other instruction-finetuning works [51, 50, 52]. This single objective allows for learning to proceed seamlessly across tasks without introducing conflicting gradients or scale issues coming from different objectives and loss functions (e.g., Cross-Entropy for classification versus Mean Squared Error for regression).

In addition, it allows one to extend the model with additional tasks in the future without requiring changes in the model architecture or training it from scratch. In summary, ChatNT provides a general genomics AI system that solves multiple tasks in a conversational manner, thus providing a new paradigm for genomics models. In addition to seamlessly integrating multiple types of labeled and experimental data into a single general foundation model, ChatNT is designed to be conversational to enable users to easily interact with it and to use it without requiring a programming background as shown in FIG. 9. ChatNT relies on a frozen English language model, Vicuna 7B [50], that has been instruction fine-tuned from LLaMA [47]. Therefore, ChatNT keeps all the intrinsic conversational capabilities of the language model. Interestingly, as the training dataset used to build LLaMA already contained a large set of life sciences papers, the agent is also capable to answer multiple questions about genomics such as defining regulatory elements like promoters and enhancers, zero shot, i.e., without any additional training data. Additionally, ChatNT can answer numerous non-biology related questions and solve tasks such as summarizing or writing simple programming code. As the approach is general and builds on top of any pre-trained English language model, ChatNT capabilities can improve organically with new and more powerful open-sourced language models. While the conversational capability is an important aspect of ChatNT, but is already provided by the respective language model, is the present example focuses on demonstrating that the conversational agent ChatNT can solve a wide range of advanced genomics tasks in English with high accuracy.

G.ii. Chatnt Shows State-of-the-Art Performance on the Nucleotide Transformer Benchmark

In order to develop ChatNT and optimize its architecture, an instructions version of the Nucleotide Transformer benchmark was created [26]. This collection of genomic datasets is suitable for fast iteration during model experimentation as it contains a varied panel of small-sized datasets and has been extensively evaluated in multiple studies of DNA foundation models [26, 29]. ChatNT was trained to solve all 18 tasks at once and in English and evaluated its performance on test set DNA sequences and questions.

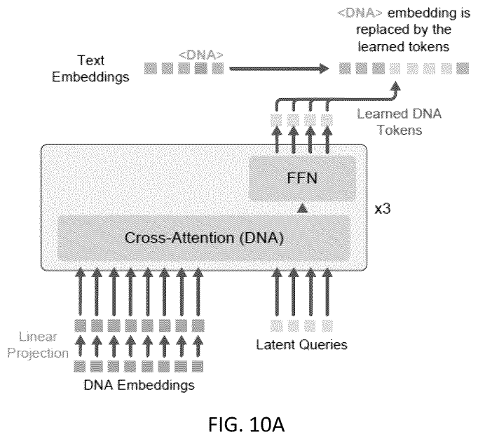

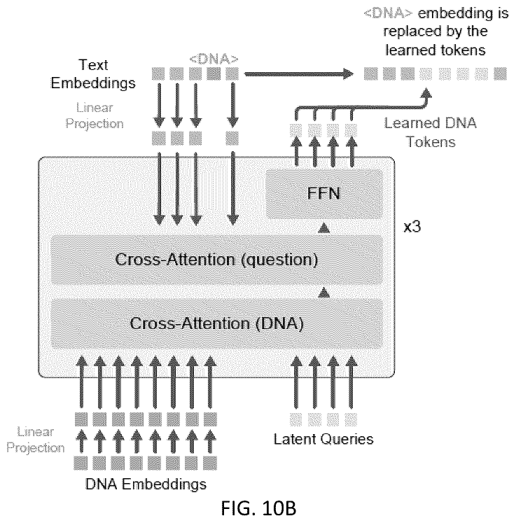

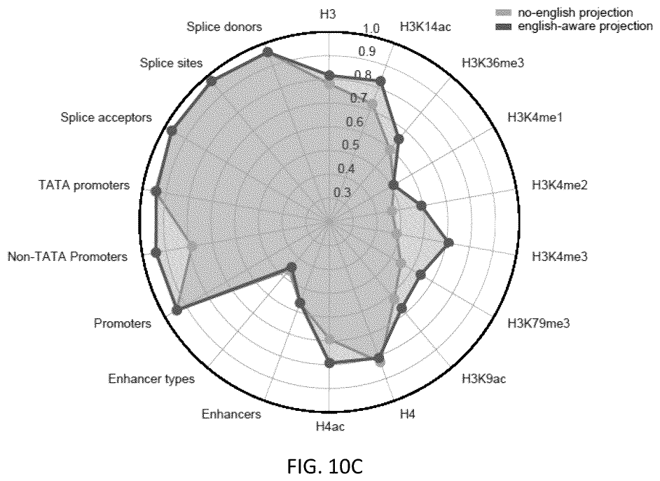

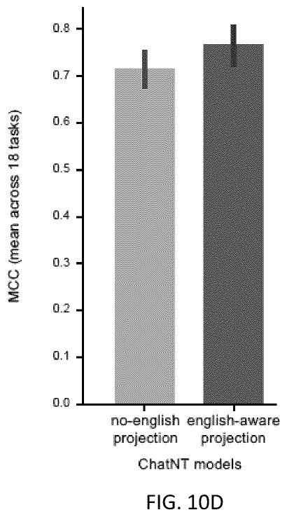

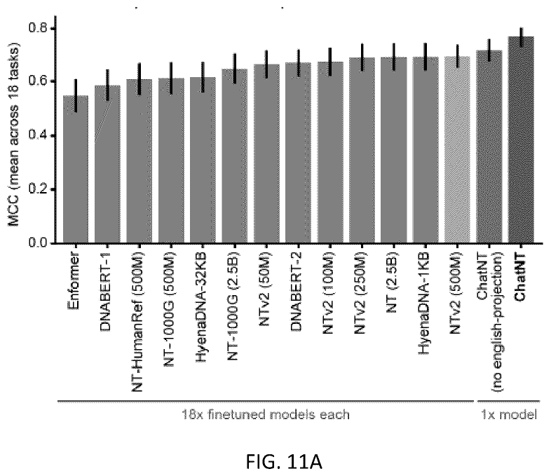

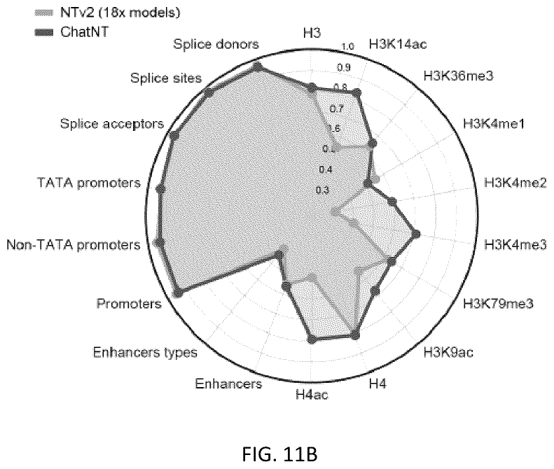

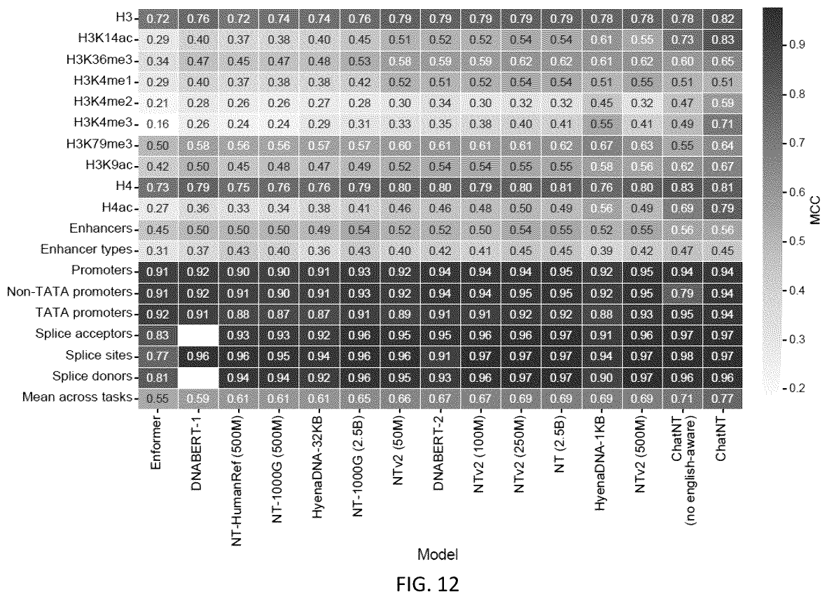

This benchmark was first used to systematically compare the performance of ChatNT with two different projection architectures. The classical way of aggregating information from the encoder in previous multimodal models is to use a trainable projection to convert the encoder embeddings into language embedding tokens, which have the same dimensionality of the word embedding space in the language model [40, 49, 41, 42]. In ChatNT, the Perceiver resampler from Flamingo [41] based on gated cross-attention was used as projection layer as shown in FIG. 10A. Using this projection layer and finetuning both the DNA encoder and the projection on all 18 tasks, ChatNT obtained a state-of-the-art accuracy on this benchmark with an average Matthew's correlation coefficient (MCC) of 0.71, 2 points above the previous state-of-the-art Nucleotide Transformer v2 (500M) model as shown in FIGS. 11A, 12.

However, similar to all other projection layers [40, 49, 53], the current implementation of the Perceiver resampler generates the same fixed set of embeddings for the encoder tokens independently of the question asked, and therefore it needs to capture in this set of embeddings all relevant information for every downstream task. Without wishing to be bound to any particular theory, a hypothesis tested herein is that this feature can create an information bottleneck in genomics when scaling the model for multiple downstream tasks given the diversity of potential sequences, from different lengths and species, and biological properties. Therefore, an English-aware Perceiver projection was developed that extracts representations from the input sequence dependent on the English question asked by the user, which allows to leverage contextual information encoded in the input DNA sequences that are relevant for the specific question as shown in FIG. 10B. Significantly improved performance was observed by accounting for the question when projecting the DNA embeddings into the English decoder space (average MCC of 0.77 vs 0.71) as shown in FIGS. 10C-10D. This can be explained by the very context- and task-specific information in DNA sequences that must be retained in order to tackle diverse genomics tasks. Since the decoder remains frozen, the projection layer not only needs to bring the sequence embeddings into the embedding space of the English decoder, but also to perform the operations to extract the relevant information from the embedding to answer the question. The results show that making the projection aware of the question facilitates both aspects thus achieving a better performance and transfer across tasks.

In summary, ChatNT with an English-aware projection (from now on just called ChatNT) achieves a state-of-the-art accuracy on this benchmark (average MCC of 0.77) in addition to solving all 18 tasks at once as shown in FIG. 11A. Strikingly, ChatNT improves the average performance by 8 points over the previous state-of-the-art Nucleotide Transformer v2 (500M) model, which was used as the DNA encoder within ChatNT (average MCC of 0.77 vs 0.69) as shown in FIGS. 11A-11B. The results demonstrate that a single unified objective formulated in natural language triggers transfer learning between multiple downstream tasks and helps deliver improved performance.

G.iii. A New Curated Genomics Instructions Dataset of Biologically Relevant Tasks

Although the Nucleotide Transformer benchmark [26] was very suitable for model experimentation and to debug the system, it misses many tasks of great biological relevance in genomics related to more complex biological processes as well as more recent experimental techniques and tasks that involve quantitative predictions. Therefore, a second genomics instructions dataset was curated containing 27 genomics tasks framed in English derived from different studies that cover several regulatory processes. These include tasks related to DNA (21 tasks), RNA (3) and protein sequences (3) from multiple species framed as both binary/multi-label classification and regression tasks. The final instructions dataset contains a total of 605 million DNA tokens, i.e., 3.6 billion base pairs, and 273 million English tokens (including an average of 1,000 question/answer pairs per task) as shown in FIG. 8B.

This collection includes a non-redundant subset of tasks from the Nucleotide Transformer [26] and the BEND [54] benchmarks, complemented with relevant tasks from the plant AgroNT benchmark [55] and human ChromTransfer [56]. These benchmarks have been extensively used in the literature, come from different research groups, and represent diverse DNA processes and species. These selected tasks include binary and multi-label classification tasks covering biological processes related to histone and chromatin features, promoter and enhancer regulatory elements, and splicing sites.

State-of-the-art and challenging regression tasks related to promoter activity [55], enhancer activity [11], RNA polyadenylation [20, 21] and degradation [19], and multiple protein properties [57] were also added. These are reference datasets in the respective fields and related to very complex properties of biological DNA, RNA and protein sequences. All RNA and protein tasks are predicted from the corresponding DNA and CDS sequences instead of the RNA and protein sequences, respectively. Codon usage complexity is a challenge for determining DNA sequences corresponding to particular protein sequences. Therefore, the CDS annotations for protein tasks curated at Boshar et al. [57] were used This challenge is not present where RNA sequences are converted to corresponding DNA sequences.

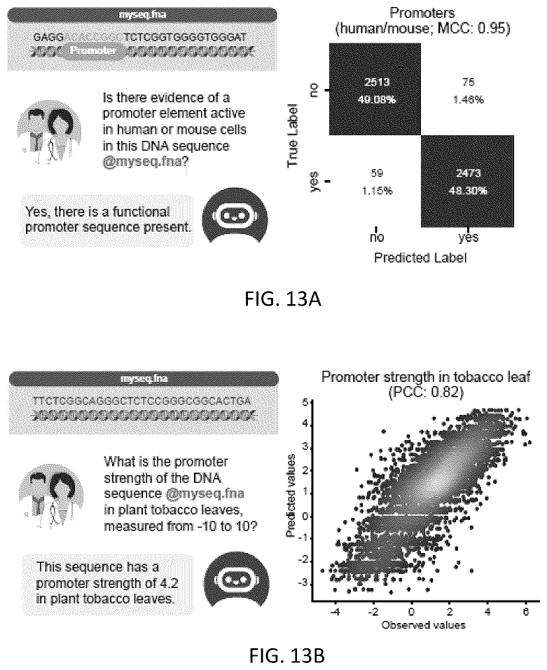

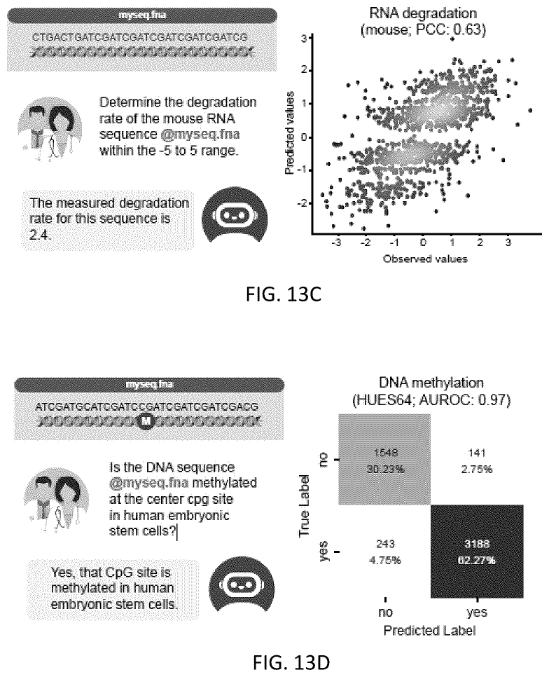

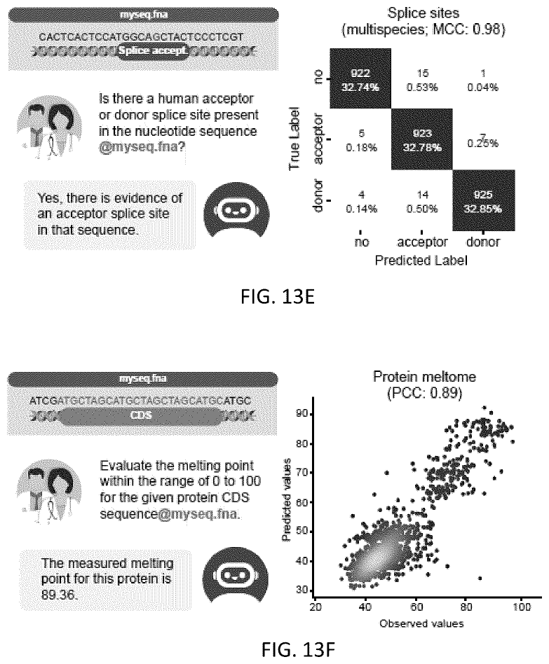

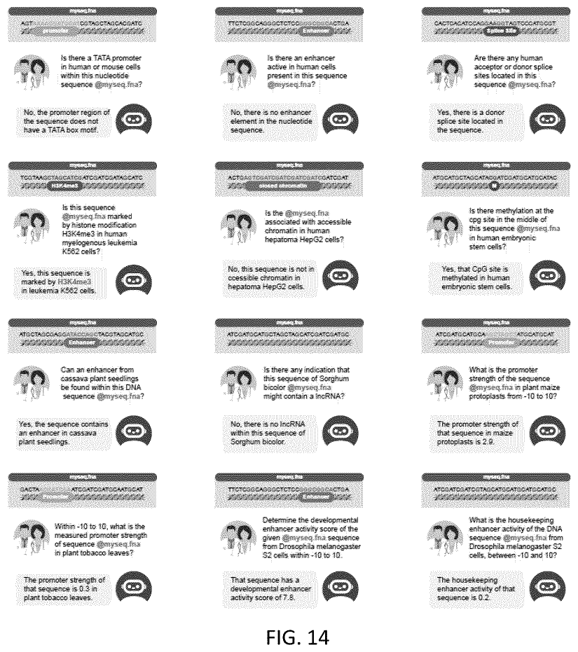

FIGS. 9, 13A-13F show examples of questions and answers for different types of genomics tasks used in the dataset as further shown in FIGS. 14-16. For instance, a training example for an enhancer classification task would be “User: Is there an enhancer from human cells present in this sequence @myseq.fna, and can you characterize as weak or strong?ChatNT: Yes, a weak enhancer is present within the DNA sequence that you provided.”, where the projected embeddings of the candidate DNA sequence are inserted at the @myseq.fna position. Regression tasks are also framed in English and the agent needs to write the digits corresponding to the requested quantity: for example, “User: Determine the degradation rate of the mouse RNA sequence @myseq.fna on a scale from −5 to 5. ChatNT: The measured degradation rate for this sequence is 2.4.” The loss is equally computed as the cross-entropy loss between the predicted and the target answer tokens. For performance evaluation, the digits from each answer were extracted and their correlation with the ground-truth values was tested.

In summary, this curated set of tasks provides a general perspective of the capabilities and usefulness of the model in different biological sequence domains. ChatNT was trained as a general agent to solve all 27 genomics tasks at once and in English, and its performance was compared with the state-of-the-art specialized model for each task.

G.iv. Chatnt Achieves High Performance on Multiple Tasks Across Different Genomics Processes and Species

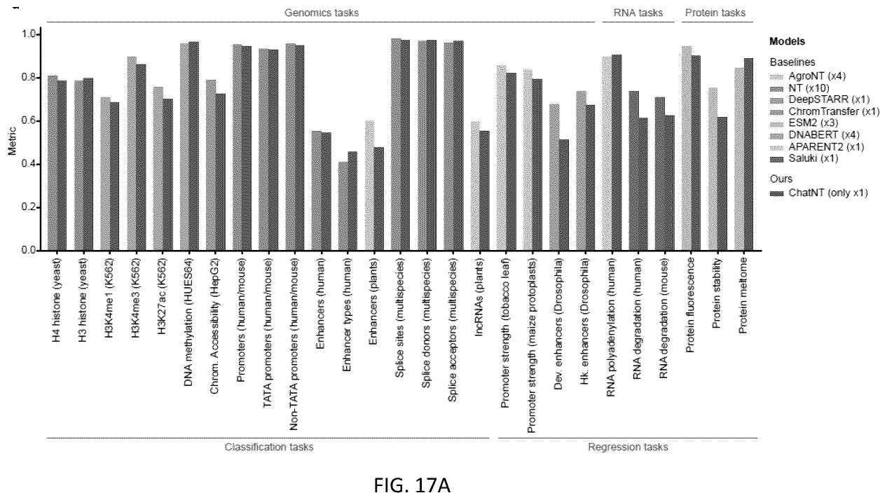

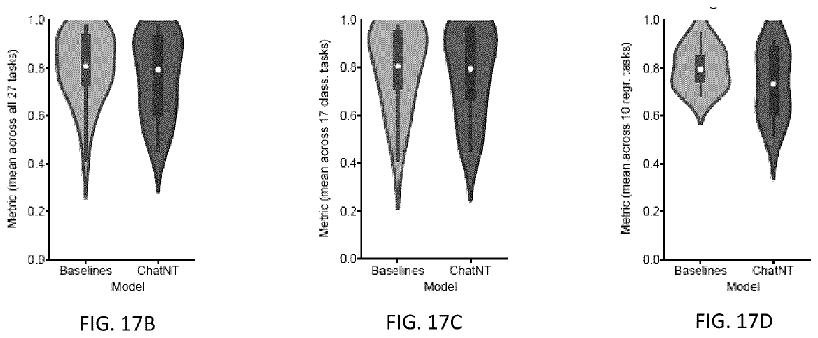

The performance of ChatNT is first evaluated on the 21 tasks related to different DNA processes from yeast, plants, fly, mouse, and human. ChatNT is competitive with the performance of the different specialized models that were fine-tuned directly on each of these individual tasks as shown in FIGS. 13A, 13B, 13D, 13E, 17A, 17C. In particular, an improved performance was obtained on the detection of human enhancer types. Still, significantly reduced performance was observed for enhancers from plant species when compared with the state-of-the-art AgroNT model fine-tuned specifically on this task. Since AgroNT was pre-trained on genomes from 48 diverse plant species, improving the encoder used in ChatNT might lead to improved performance on this type of tasks.

As ChatNT solves the tasks in English, it can seamlessly handle binary and multi-label classification tasks. By extracting the term predicted by ChatNT in the answer, its predictive performance can be quantified. ChatNT accurately identifies input sequences with human or mouse promoters as shown in FIG. 13A, with CpG sites methylated in human embryonic stem cells (HUES64 cell line) as shown in FIG. 13D, and with splice acceptor and donor sites as shown in FIG. 13E.

ChatNT is also able to solve quantitative tasks by writing the digits of the predicted score. Competitive performance was observed on predicting promoter activity in plants, namely tobacco leaves as shown in FIG. 13B and maize protoplasts, but significantly reduced performance was observed on Drosophila enhancer activity over the state-of-the-art DeepSTARR model as shown in FIGS. 17A-17D. Importantly, the distributions of the predicted digits correlate well with the original scores as shown in FIG. 13B. This capability to proficiently address regression tasks is of importance in biology, and is particularly significant in light of the acknowledged limitations and unreliability of numerical processing in language models [58, 59]. Still, a reduced average performance was observed on regression tasks over classification ones, likely due to the difference in complexity and classification tasks being more represented in the training set. This might be solved by improving the balance between classification and regression tasks during training, through either a weight loss or a task sampling frequencies curriculum [60].

G.v. Chatnt Solves Transcriptomics and Proteomics Tasks

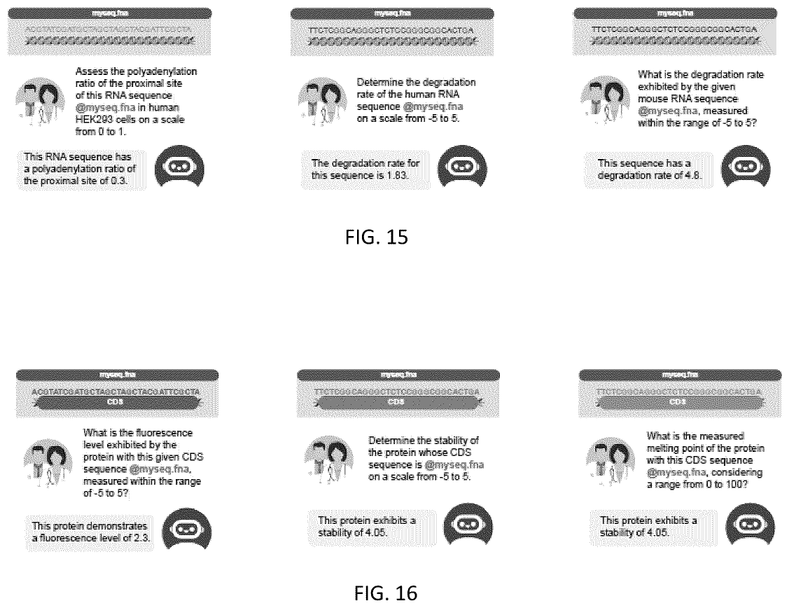

ChatNT is built with a flexible architecture that allows it to handle any type of biological sequence that can be processed with the DNA encoder, the Nucleotide Transformer[26]. To showcase its generalization, the new genomics instructions dataset included three RNA and three protein regression tasks as shown in FIGS. 15-16. These include predicting RNA polyadenylation and degradation rates as well as different protein features. Examples of conversations used for model training are: “User: What is the measured polyadenylation ratio of the proximal site of the RNA sequence @myseq.fna in human HEK293 cells, considering a range from 0 to 1?ChatNT: That sequence has a polyadenylation ratio of the proximal site of 0.69.” and “User: Specify the melting point of the protein with the given coding sequence (CDS) @myseq.fna within the 0 to 100 range. ChatNT: This protein demonstrates a melting point of 80.81.” The performance of ChatNT was compared to the state-of-the-art specialized models APARENT2 for polyadenylation [21], Saluki for RNA degradation [19], and ESM2 for the protein tasks [48].

Overall, good performance was observed for ChatNT on the test sets of the 6 RNA and protein tasks, with Pearson correlation coefficients (PCCs) between 0.62 and 0.91 as shown in FIGS. 13C, 13F, 17A. ChatNT outperformed the specialized models for the prediction of proximal polyadenylation site ratio (PCC of 0.91 vs 0.90) and protein melting points (PCC of 0.89 vs 0.85). Regarding the RNA degradation tasks in human and mouse, ChatNT obtained a PCC of 0.62 and 0.63, ten points below the specialized Saluki model [19](PCC of 0.74 and 0.71). ChatNT also obtained competitive performance with the state-of-the-art protein language model ESM2 [48] on the two other protein tasks related to protein fluorescence and stability. Although ChatNT cannot yet outperform every specialized model on RNA and protein tasks, it can handle such tasks and achieve high performance using the DNA foundation model Nucleotide Transformer as a DNA encoder. ChatNT's flexible architecture allows to plug-in different encoders, such as language models specialized for RNA [61, 62, 63, 64] and protein domains [48], which should reduce the gap to specialized deep learning models in the transcriptomics and proteomics fields and improve the capabilities and generalization of ChatNT towards a unified model of biology.

G.vi. Assessing the Confidence of ChatNT Answers

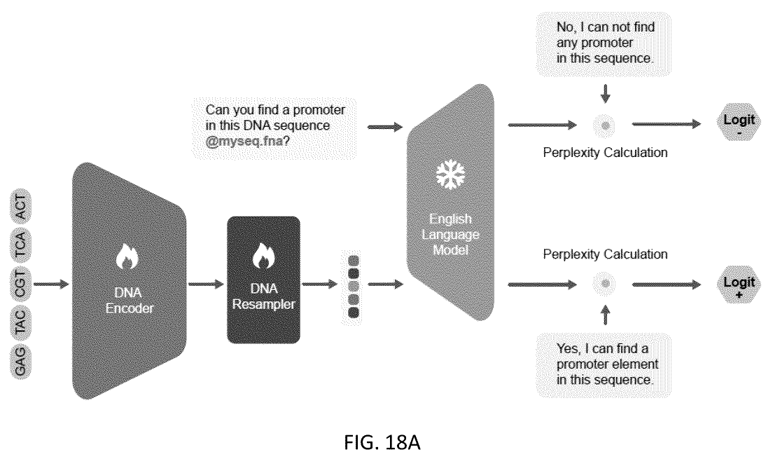

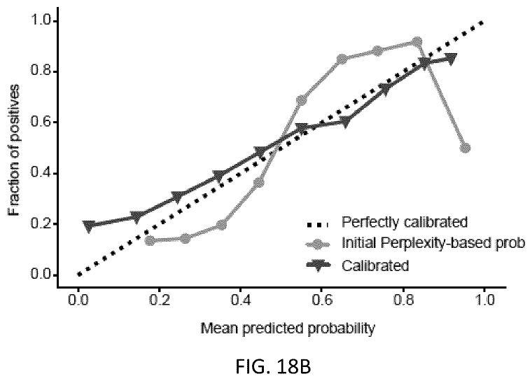

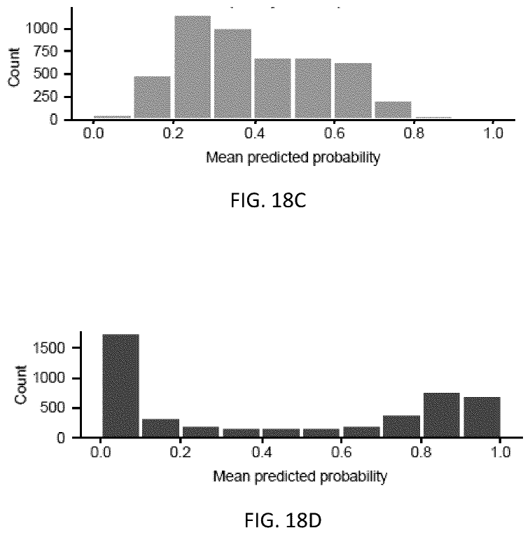

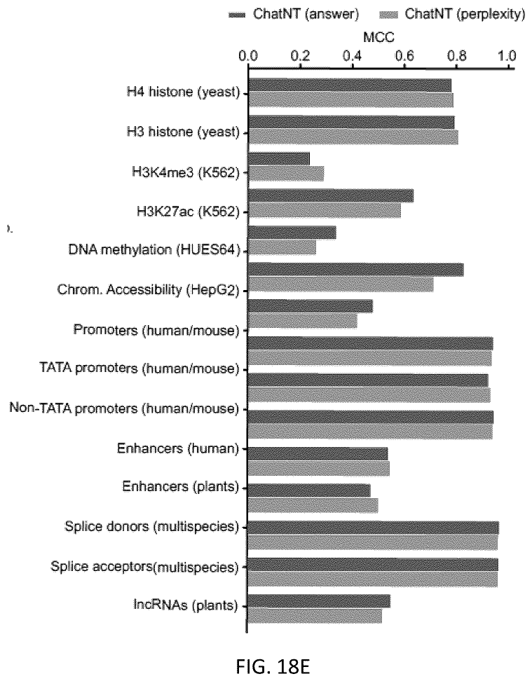

ChatNT is built to assist and augment scientists and researchers in their daily research. As such, its performance and reliability are paramount. However, in contrast to standard machine learning models that return probabilities or quantitative scores, ChatNT directly answers questions, preventing the user to get a sense of its confidence and thus reducing its practical value for sensitive applications. This is an important challenge and common to all current conversational agents [38, 39, 40]. To address this, a way to assess the confidence of the agent for binary classification tasks was introduced. Instead of generating directly answers to the binary classification question for a given sequence, the model perplexity is computed for that question over examples of both positive and negative answers. These selected answers are not included in the model training dataset. Those perplexity values towards positive and negative answers are then used to derive logits and probabilities for each class for the candidate question. This method allows one to derive probabilities from ChatNT for each question example, similar to standard classifiers, and it is referred as perplexity-based classifier as shown in FIG. 18A.

Computing probabilities enables to assess the calibration of the model, i.e., the correlation between the predicted probability, its confidence, and the accuracy of its prediction. A model is well calibrated when a prediction of a class with confidence p is correct 100p % of the time. The ChatNT perplexity-based probabilities were computed for all binary classification tasks. FIGS. 18B, 18D show an example of a calibration plot based on the predictions for the chromatin accessibility task. The model is well calibrated for low- and high-confidence areas, but less in medium-confidence ones. For instance, examples predicted with a probability of 0.9 are correctly predicted 90% of the time while examples predicted with probability 0.5 are correctly predicted only 25% of the time. To improve this, the model can be calibrated by fitting on the training set a Platt's model [65], to improve the confidence of the model across all ranges of predictions as shown in FIGS. 18B-18D. This calibration step is performed for all binary classification tasks. Overall, the same performance for ChatNT is achieved across tasks using these perplexity-based predictions as show in FIG. 18E but with improved calibration. As a consequence, the approach can accurately measure the predictive performance of a language model in addition to effectively assessing its uncertainty level. This technique, while being general, should also be beneficial to other language model fields.

G.vii. Discussion

ChatNT is presented as the first (to the best of knowledge of inventors) multimodal conversational agent that can handle DNA, RNA and protein sequences and solve multiple biologically relevant downstream tasks. The first datasets of genomics instructions tasks are built and curated that include binary and multi-labels classification and regression tasks spanning different species and genomics processes. Tasks relative to transcriptomic and proteomic processes were also included to demonstrate the versatility and generality of this approach across domains. ChatNT achieves a new state-of-the-art on the Nucleotide Transformer benchmark [26] and demonstrates a performance on par with specialized models on the new set of 27 tasks. Importantly, unlike conventional approaches requiring a specialized model for each task, ChatNT solves all tasks within a unified model in addition to offering a simple and natural chatbot interface for people to use the model. A technique to probe the confidence of language models for binary classification tasks is introduced and used to calibrate the models when needed. Altogether, the ChatNT implementation described in this example demonstrates that natural language LLMs can be extended to process bio-sequence modalities, displaying not only conversational capabilities but also answering accurately multiple biologically relevant questions.

To extract the complex information from DNA sequences that is needed to solve all tasks in a single unified model, an architecture based on the Perceiver resampler [41] is introduced to resample and project DNA embeddings into the natural language embedding space. An information bottleneck issue was identified that arises from the diversity of tasks, species and biological processes encoded in DNA sequences, and it was shown how to solve it by conditioning the projection on the question asked. This conditioning allows the projection module to extract from the DNA embeddings the right amount of information to solve the task at hand, as is shown by the improved performance over a projection module that is not conditioned on the question.

Herein, ChatNT focuses on situations where a user, such as a researcher or scientist, is interested in detecting molecular phenotypes or computing quantitative properties for a given DNA sequence. While this encompasses an already significant number of practical use-cases, it would be interesting to expand the agent capabilities to handle other typical bioinformatics pipelines. Such pipelines could include calling tools to compute statistics about the sequences, aligning the sequences to a reference database to compute multiple sequence alignments, query external databases for additional information about the sequences, or to recursively call the ChatNT model over a FASTA file containing multiple sequences and generating a summarized table results with its corresponding analysis. This is supported by the success of external tools in large language models such as Toolformer [66], LLaVA-Plus [49], geneGPT [67] or GPT-4 [39]. Such pipelines could also benefit from ChatNT's capability to handle several sequences at the same time in order to reduce the inference compute cost. Replacing ChatNT's current English decoder by larger models and/or models finetuned using Reinforcement Learning Human Feedback (RLHF) such as Llama2-chat 70B [52] could also help extending the model capabilities in these directions as well as improving its overall usefulness.

The capabilities of ChatNT have been demonstrated for DNA sequences using a pre-trained DNA foundation model, the Nucleotide Transformer [26]. As shown in the experiments, working with DNA sequences allows to tackle tasks not only in genomics but also transcriptomics and proteomics, the latter using the corresponding CDS region. However, the approach could be easily extended to integrate encoders from other omics modalities such as RNA [61, 62, 63, 64] and protein [48, 68] language models to work natively with RNA and amino acid sequences. Through the positional tag system that supports multiple sequences, one could simply add an arbitrary number of encoders and train their respective projections to combine different omics and modalities within the same questions. Such approach could expand even further the capabilities and performance of the model by achieving superior transfer learning across modalities.

This work demonstrates that it is possible to build multimodal bio-sequence/English conversational agents that can solve advanced, biologically relevant tasks, and is meant to lay a first set of foundations to build future highly-capable agents that understand biological sequences and principles. Similar to the developments in NLP [69, 52, 70, 71] and multimodal models [72], it is expected that new capabilities, such as zero-shot performance, to emerge through developments on two main fronts: (1) scaling the number of tasks by including examples from diverse biological processes, tissues, individuals and species [73, 74]; and (2) integrating more data modalities, such as RNA and protein sequences, imaging data and health records from individuals. When such capabilities emerge, it will be of the highest importance to carefully assess model safety and robustness, for instance through red teaming [75]. As such, ChatNT represents an important step along the trajectory towards general purpose AI for biology and medicine [76].

G.viii. Methods: ChatNTModel

### Architecture

The ChatNT is a multimodal agent that takes as input one or multiple DNA sequences and an English prompt and returns a distribution over English words that is used to auto-regressively produce an answer in English. A DNA English token placeholder <DNA> is introduced that is added in the input English prompt for the user to refer to the DNA sequence. The architecture is also extended to handle several DNA sequences. In this case, each DNA sequence is processed independently by the DNA encoder and the input English prompt is expected to contain as many DNA English token placeholders as sequences are inputted.

The ChatNT architecture is made of three parts: a pre-trained DNA encoder, a projection model that projects the DNA embeddings into the English tokens embedding spaces and a pre-trained English decoder. While the architecture is general and could work with any choice of DNA Encoder and English decoder, it was decided to use the pre-trained Nucleotide Transformer v2 (500M parameters) [26] and Vicuna-7b (instruction fine-tuned Llama model with 7B parameters) [50] models, respectively. During training, the English decoder was kept frozen and only the weights of the DNA encoder and the projection model were updated. The projection model is initialized from scratch at the beginning of the training.

The DNA Encoder processes the DNA sequence and returns one embedding vector per input token, one token representing a nucleotide 6-mers in the case of the Nucleotide Transformer model. L is the number of nucleotides in the DNA sequence and N is the number of DNA tokens (with roughly N≈L/6). Every input DNA sequence was padded if needed until a final length of 2,048 tokens, representing approximately 12kb. As the output embedding dimension of the DNA encoder can be different from the words embedding dimensions of the English language model, a dense neural network is first used to project each DNA token embedding to the English word dimension. In a second phase, a Perceiver resampler architecture [41] that uses cross-attention between the projected DNA tokens embeddings and learnable queries, to re-sample the N DNA tokens embedding to K embedding vectors as shown in FIG. 11A was used. This Perceiver resampler was adapted to include an additional cross-attention step between the learnable queries and the English question in order to extract context-dependent representations from the DNA sequence as shown in FIG. 11B.

On the other hand, the English prompt is tokenized, and English tokens embeddings are produced for each tokens. The K resampled DNA embedding vectors are then inserted in place of the DNA sequence placeholder tokens in the English input sequence. In the case of multiple input DNA sequences, these operations are applied consecutively and independently for each DNA sequence. Several values of K were used, observing that low values such as 1 or 4 are not enough for the DNA encoder to impact the behavior of the frozen English decoder. The value of K=64 was found to provide a good trade-off between the input length of the English decoder and the performance in practice.

During inference, the DNA encoder embeddings for the DNA sequences are computed only once. The inference is done autoregressively by predicting sequentially each new token until an end of sequence token is predicted. The key, queries and values of the English decoder are cached during generation to avoid computing unnecessary operations. Temperature sampling with a temperature of r=0.001 was used.

The whole codebase of the ChatNT has been developed in Jax [77] using Haiku [78] for neural networks implementation. All trainings were performed on a cluster of 8 GPU H100 instances and evaluations of the model can be done in a single GPU A100-80gb. All trained parameters from the DNA encoder and perceiver projection as well as optimizer accumulators and all frozen parameters from the English decoder are stored and updated in float32.

### Training

ChatNT was trained using Adam optimizer [79] with 1r=3e-5 and default settings for other hyperparameters: β₁=0.9, β₂=0.999, ϵ=1e-8, ϵᵣₒₒₜ=0.0. A gradient clipping of 1 was used and gradients over a batch size of 65,536 tokens were accumulated, equivalent to 256 samples. A uniform sampling was used over tasks per batch such that each batch has the same proportion of samples per task. The model was trained on the 27-task dataset for 2B tokens (7.8M samples) on a cluster of 8 GPU H100 over 4 days.

### Hyperparameters

Below all hyperparameters for the different parts of ChatNT are described.

| | | Perceiver | English |
|---|---|---|---|
| | DNA encoder | Resampler | decoder |
| Number of layers | 29 | 3 | 32 |
| Number of heads | 16 | 20 | 32 |
| Embedding | 1024 | 4096 | 4096 |
| dimension | | | |
| Feed forward | 4096 | 11008 | 11008 |
| dimension | | | |
| Activation type | swish | GeLo | swiGlu |
| Positional | RoPe | RoPe | RoPe |
| encoding type | | | |
| Total number | 500M | 800M | 7B |
| of parameters | | | |
| Input tensor shape | (1, 2048) | (1, 2048, 1024) | (1, 1024, 4096) |
| Output tensor | (1, 2048, | (1, 64, 4096) | (1, 1024, 32000) |
| shape | 1024) | | |
| Float Precision | Float32 | Float32 | Float32 |
| Initialization | Pre-trained | From scratch | Pre-trained |
| | (NT-v2- | | (vicuna-7b) |
| | 500m) | | |
| Update | Updated | Updated | Frozen |

### Evaluation

Evaluating the performance of ChatNT can be done in a single GPU A100 in batches of 32 samples and takes from 1 to 40 minutes to generate a maximum of 40 tokens per sample (13 tokens per second). For each task, ChatNT was evaluated on upmost 5,000 sampled test samples and the metric used in the respective benchmark study was reported.

G.ix. Methods: Genomics Instructions Datasets

### Instructions for the Nucleotide Transformer Benchmark

An instructions version of the Nucleotide Transformer benchmark was created as shown in Table G1[26]. To convert the DNA sequence datasets into instructions datasets, dozens of English questions and answers were curated for each task and a question/answer pair per input DNA sequence was sampled. The DNA token placeholder <DNA> was used in the question when referring to the input DNA sequences. The answer contains the classification label for the respective input sequence. All 18 binary/multi-label classification datasets were converted into diverse question/answer instructions for each DNA sequence. For each task, train and test sets were provided containing different DNA sequences as well as different questions to assess the performance and English generalization capabilities of the model. The same train and test sets as the original dataset were kept.

TABLE G1
Information about all tasks in the Nucleotide Transformer benchmark.

| Task | Biological | | No. | Sequence | N | Dataset |
|---|---|---|---|---|---|---|
| name | process | Species | classes/regression | length (bp) | (train/test) | source |
| H3 histone | Histones | yeast | 2 | 500 | 13140/1461 | NT-benchmark |
| H4 histone | Histones | yeast | 2 | 500 | 13468/1497 | NT-benchmark |
| H3K4me1 | Histones | yeast | 2 | 500 | 28509/3168 | NT-benchmark |
| H3K4me2 | Histones | yeast | 2 | 500 | 27614/3069 | NT-benchmark |
| H3K4me3 | Histones | yeast | 2 | 500 | 23953/2884 | NT-benchmark |
| H3K9ac | Histones | yeast | 2 | 300 | 25003/2779 | NT-benchmark |
| H3K14ac | Histones | yeast | 2 | 300 | 29743/3303 | NT-benchmark |
| H3K36me3 | Histones | yeast | 2 | 500 | 31392/3488 | NT-benchmark |
| H3K79me3 | Histones | yeast | 2 | 500 | 25953/2884 | NT-benchmark |
| H4ac | Histones | yeast | 2 | 500 | 30685/3410 | NT-benchmark |
| Promoters | Promoters | human/mouse | 2 | 300 | 53276/5920 | NT-benchmark |
| TATA promoters | Promoters | human/mouse | 2 | 300 | 5509/621 | NT-benchmark |
| Non-TATA promoters | Promoters | human/mouse | 2 | 300 | 47767/5299 | NT-benchmark |
| Splice sites | Splice sites | human/mouse/rat/fly/zebrafish | 3 | 400 | 27000/3000 | NT-benchmark |
| Splice donors | Splice sites | 147 species | 2 | 600 | 19775/2198 | NT-benchmark |
| Splice acceptors | Splice sites | 147 species | 2 | 600 | 1996 [symbol] /2218 | NT-benchmark |
| Enhancers | Enhancers | human | 2 | 200 | 14968/400 | NT-benchmark |
| Enhancer types | Enhancers | human | 3 | 200 | 14968/400 | NT-benchmark |

[symbol]

indicates data missing or illegible when filed

### New Curated Genomics Instructions Dataset of Biologically Relevant Tasks

The new genomics instructions dataset created here contains a set of 27 tasks framed in English derived from different studies as shown in Table X2. It covers several regulatory processes related to DNA (21 tasks), RNA (3) and protein sequences (3). These tasks are derived from multiple species, including human, mouse, fly and plants. Among all tasks there are 15 binary classification, 2 multi-label classification and 10 regression tasks. The number of training examples per task ranges from 5.5K to 3M.

TABLE G2
Information about all tasks and respective baseline performance and metrics.

| Task | Biological | | | No. | Sequence | N | | Model | Baseline | Dataset |
|---|---|---|---|---|---|---|---|---|---|---|
| name | process | Category | Species | classes/regression | length (bp) | (train/test) | [symbol] | baseline | performance | source |
| [symbol] | Histones | [symbol] | yeast | [symbol] | [symbol] | [symbol] | [symbol] | [symbol] | [symbol] | NT- |
| | | | | | | | | | | benchmark |
| [symbol] | Histones | Genomics | yeast | [symbol] | [symbol] | [symbol] | [symbol] | [symbol] | [symbol] | NT- |
| | | | | | | | | | | benchmark |
| [symbol] | Histones | Genomics | human | [symbol] | [symbol] | [symbol] | [symbol] | [symbol] | [symbol] | [symbol] |
| [symbol] | Histones | Genomics | human | [symbol] | [symbol] | [symbol] | [symbol] | [symbol] | [symbol] | [symbol] |
| [symbol] | [symbol] | Genomics | human | [symbol] | [symbol] | [symbol] | [symbol] | [symbol] | [symbol] | [symbol] |
| [symbol] | DNA [symbol] | Genomics | human | [symbol] | [symbol] | [symbol] | [symbol] | [symbol] | [symbol] | [symbol] |
| [symbol] | DNA [symbol] | Genomics | human | [symbol] | [symbol] | [symbol] | [symbol] | [symbol] | [symbol] | [symbol] |
| [symbol] | Promoters | Genomics | [symbol] | [symbol] | [symbol] | [symbol] | [symbol] | [symbol] | [symbol] | NT- |
| | | | | | | | | | | benchmark |
| [symbol] | Promoters | Genomics | [symbol] | [symbol] | [symbol] | [symbol] | [symbol] | [symbol] | [symbol] | NT- |
| | | | | | | | | | | benchmark |
| [symbol] | Promoters | Genomics | [symbol] | [symbol] | [symbol] | [symbol] | [symbol] | [symbol] | [symbol] | NT- |
| | | | | | | | | | | benchmark |
| [symbol] | Enhancers | Genomics | [symbol] | [symbol] | [symbol] | [symbol] | [symbol] | [symbol] | [symbol] | NT- |
| | | | | | | | | | | benchmark |
| [symbol] | Enhancers | Genomics | [symbol] | [symbol] | [symbol] | [symbol] | [symbol] | [symbol] | [symbol] | NT- |
| | | | | | | | | | | benchmark |
| [symbol] | Enhancers | Genomics | [symbol] | [symbol] | [symbol] | [symbol] | [symbol] | [symbol] | [symbol] | [symbol] |
| [symbol] | Splice | Genomics | [symbol] | [symbol] | [symbol] | [symbol] | [symbol] | [symbol] | [symbol] | NT- |
| | sites | | | | | | | | | benchmark |
| [symbol] | Splice | Genomics | [symbol] | [symbol] | [symbol] | [symbol] | [symbol] | [symbol] | [symbol] | NT- |
| | sites | | | | | | | | | benchmark |
| [symbol] | Splice | Genomics | [symbol] | [symbol] | [symbol] | [symbol] | [symbol] | [symbol] | [symbol] | NT- |
| | sites | | | | | | | | | benchmark |
| [symbol] | [symbol] | Genomics | [symbol] | [symbol] | [symbol] | [symbol] | [symbol] | [symbol] | [symbol] | [symbol] |
| [symbol] | [symbol] | Genomics | [symbol] | Regression | [symbol] | [symbol] | [symbol] | [symbol] | [symbol] | [symbol] |
| [symbol] | [symbol] | Genomics | [symbol] | Regression | [symbol] | [symbol] | [symbol] | [symbol] | [symbol] | [symbol] |
| [symbol] | [symbol] | Genomics | [symbol] | Regression | [symbol] | [symbol] | [symbol] | [symbol] | [symbol] | [symbol] |
| [symbol] | [symbol] | Genomics | [symbol] | Regression | [symbol] | [symbol] | [symbol] | [symbol] | [symbol] | [symbol] |
| [symbol] | [symbol] | [symbol] | [symbol] | Regression | [symbol] | [symbol] | [symbol] | [symbol] | [symbol] | [symbol] |
| [symbol] | [symbol] | [symbol] | [symbol] | Regression | [symbol] | [symbol] | [symbol] | [symbol] | [symbol] | [symbol] |
| [symbol] | [symbol] | [symbol] | [symbol] | Regression | [symbol] | [symbol] | [symbol] | [symbol] | [symbol] | [symbol] |
| [symbol] | [symbol] | [symbol] | [symbol] | Regression | [symbol] | [symbol] | [symbol] | [symbol] | [symbol] | — |
| [symbol] | [symbol] | [symbol] | [symbol] | Regression | [symbol] | [symbol] | [symbol] | [symbol] | [symbol] | [symbol] |
| [symbol] | [symbol] | [symbol] | [symbol] | Regression | [symbol] | [symbol] | [symbol] | [symbol] | [symbol] | [symbol] |

[symbol]

indicates data missing or illegible when filed

The DNA sequence datasets were converted into instructions datasets as described above for the Nucleotide Transformer benchmark. The answer contains the classification label or regression score (up to decimal cases) for the respective input sequence. In addition to simple examples with a single turn of question/answer with a single sequence, more complex examples with multiple turns with consecutive questions that can be related or not, and exchanges where the question refers to multiple sequences were also added. The final genomics instructions dataset contains a total of 605 million DNA tokens, i.e., 3.6 billion base pairs, and 273 million English tokens (including questions and answers).

For each task, train and test sets were obtained containing different DNA sequences as well as different questions to assess the performance and English generalization capabilities of the model.

### Baselines for the Genomics Tasks

For each of the 27 genomics tasks, the performance of ChatNT was compared with the state-of-the-art method for the respective dataset. These included the convolutional neural networks DeepSTARR [11], ChromTransfer [56], APARENT2 [21] and Saluki [19]; and the fine-tuned foundation models based on Nucleotide Transformer [26], agroNT [55], DNABERT [27] and ESM2 [48]. Different performance metrics per task were used to follow the same metric used in the respective studies. Details on the baseline method and performance metric per task can be found in Table X2. Most baseline performance metrics were directly retrieved from the respective papers. Only for ESM2, baseline performance metrics had to be rerun on the updated dataset versions.

G.x. Methods: Calibration of ChatNT Predictions

An approach to assess and calibrate the confidence of ChatNT answers for binary classification tasks was developed.

For a given binary classification task, N examples of positive and negative answers each are selected from the respective task's test set. These examples are denoted as yᵢ^{pos} and yᵢ^{neg}, respectively, where 0≥i>N. Then, for a given question x and DNA sequence s, the average perplexity of the model is computed over the positive and negative examples respectively. These two values are denoted as pp_{θ}^{pos}(x,s) and pp_{θ}^{neg}(x,s), respectively, where θ represents the ChatNT weights tensor that are computed as follow:

$${pp}_{θ}^{p o s} ( x , s ) = \frac{1}{N} {\sum }_{i = 0}^{N − 1} exp ( {\sum }_{j} {p}_{θ} ( {( {y}_{i}^{pos} )}_{j} \vert ( x , s , {y}_{i}^{pos} ) ) log ( {p}_{θ} ( {( {y}_{i}^{pos} )}_{j} \vert \text{} ( x , s , {y}_{i}^{pos} ) ) ) )$$

where (yᵢ^{pos})ⱼ denotes the j-th token of answer yᵢ^{pos} and p_{θ}((yᵢ^{pos})ⱼ|(x, s,yᵢ^{pos})) returns the probability of token j given the question, DNA sequence and tokens from the answers up to the j-th one according to ChatNT. The negative perplexity values are computed similarly over negative answers.

Those perplexity values towards positive and negative answers represent a measure of how well the model aligns the question to those answers. The values are interpreted directly as logits and a softmax transformation is used to compute probabilities for the respective class for the input question. This method allows to derive probabilities from ChatNT for each question example. This approach is applied to 1,000 test examples per task.

To calibrate those predictions, perplexity-based probabilities are first computed to 10,000 training examples as the calibration dataset and then these probabilities are used to fit a Platt's model [65]. More specifically, logistic regression from scikit-learn[80] is used as the calibrator model and is trained with the following parameters with an inverse regularization factor C=0.1 and with the lbfgs solver. The logistic regression model learns to map the perplexity-based probabilities from ChatNT onto a more accurate scale. This model is then applied to calibrate the probabilities of the 1,000 test examples mentioned above.

As metrics, both Area under the ROC Curve (AUROC) and MCCs are computed for both the original perplexity-based probabilities and the calibrated ones.

G.xi. Genomics Instructions Dataset

Histone modifications. As representatives for yeast histone datasets, the presence of H3 and H4 histones along the yeast genome derived from Chip-Chip experiments were used as tasks [81]. The processed data for the yeast H3 and H4 tasks was retrieved from the Nucleotide Transformer benchmark [26]. MCC was used as performance metric per histone type.

As representatives for human histone datasets, the abundance of the histone modifications H3K4mel, H3K4me3 and H3K27ac along the human genome in the model cell line K562 was used. Training and test DNA sequences and respective positive and negative labels were obtained from the BEND benchmark study [54]. Each input sequence is of length 512 bp and is assigned a positive label if a histone bound to it carries the respective mark. The size of the dataset was reduced for practical reasons by downsampling the negative sequences to twice the number of positive sequences. AUROC was used as performance metric per histone modification.

Chromatin accessibility. An example of a chromatin accessibility prediction task was retrieved from ChromTransfer [56], selecting data from the cell line HepG2 since it was the most challenging task in the dataset. Their fine-tuning dataset was used based on ENCODE data with input sequences of 600 bp. Positive sequences were defined as regions that were only accessible in that cell line among the six cell lines considered in the study (n=31,211 for HepG2), while negatives (n=54,995) were sampled from the positives of the other cell lines and other regulatory regions from ENCODE. The F1 score was used as performance metric.

DNA methylation. DNA methylation processed data was collected for the human embryonic cell line HUES64 from the BEND benchmark study [54]. Each input sequence is of length 512 bp and contains a CpG site at the center that is either methylated or not. Similarly to histone marks, the size of the dataset was reduced by downsampling the negative sequences to twice the number of positive sequences. AUROC was used as performance metric similar to the BEND benchmark.

Human and mouse regulatory elements. The dataset of human and mouse promoter sequences used in the Nucleotide Transformer benchmark [26] was retrieved, originally derived from DeePromoter [82]. Sequences of 300 bp that span 249 bp upstream and 50 bp downstream of transcription start sites were considered. This resulted in 29,597 promoter regions, of which 3,065 contain and 26,532 do not contain a TATA-box motif. The same negative sets were used, ending up in a total of 59,194 sequences. These sequences were used for three different binary classification datasets: classifying sequences as promoters (NT promoter all), promoters without a TATA-box motif (NT promoter no tata), and promoters with a TATA-box motif (NT promoter tata).

For human enhancer prediction tasks, the enhancer dataset from the Nucleotide Transformer benchmark [26] was used [83]. This dataset contains enhancer (strong or weak) and nonenhancer sequences of 200 bp each. Two tasks were derived from this dataset: a binary classification task for predicting enhancers (strong and weak combined; NT enhancers) and a multi-label classification task for classifying a sequence as a strong enhancer, weak enhancer or not an enhancer (NT enhancer types). Each dataset contained 14,968 training sequences and 400 test sequences.

Multi-species splice sites. The splice site prediction tasks were collected from the Nucleotide Transformer benchmark [26]. These were based on two original datasets.

A dataset originally from SpliceFinder [84] was used that contains a training set (n=27,000) of 400 bp sequences that contain donor, acceptor, or non-splice sites detected in human genes. The test set (n=3,000) contains similar types of sequences from human but also additional species: mouse, rat, fly and zebrafish. This dataset was transformed in a multi-label classification task with labels being acceptor, donor or none (NT splice sites all).

Two additional binary classification tasks were used for the predictions of donor (NT splice sites donors) or acceptor (NT splice sites acceptors) splice sites. This task was derived from the Spliceator dataset [16], based primarily on the G3PO database, which included sequences from 147 phylogenetically diverse organisms (ranging from protists to primates, including humans). All sequences were 600 bp and were labeled as positive if they included a splicing site at the center (i.e., an acceptor or donor site, respectively). The NT splice sites donors dataset contained 19,775 training and 2,198 test sequences while the NT splice sites acceptors dataset contained 19,961 training and 2,218 test sequences.

Plant enhancers. The binary classification task for predicting enhancers in the cassava plant (Manihot esculenta) seedlings was retrieved from the AgroNT benchmark [55]. This is a balanced and GC-matched dataset of 1000 bp sequences that contain or do not contain enhancers. Sequences from every chromosome except 9 and 17 were used for training (n=16,852) while sequences from the chromosome 17 were used for testing (n=812).

Plant lncRNAs. For the binary classification task of predicting plant long non-coding RNAs (lncRNA), the dataset of Sorghum bicolor from the AgroNT benchmark was used[55]. This dataset contains lncRNA sequences with a length smaller than 6,000 bp labelled as positives and length- and GC-matched mRNA sequences labelled as negatives. The same training (8,654) and test (734) sets were used.

Plant promoter strength. The promoter strength dataset from plants was derived from the AgroNT benchmark [55]. This dataset contains 170 bp promoter sequences from three different plant species whose strength was tested in tobacco leaves and maize protoplasts. The resultant quantitative values were used for the two different promoter strength regression tasks.

Enhancer activity. For tasks related to enhancer activity, the DeepSTARR dataset was used[11]. The dataset is composed of 484,052 DNA sequences of length 249 bp, each measured for their quantitative enhancer activity towards a developmental or a housekeeping promoter in Drosophila melanogaster fruitfly S2 cells. These two measures were used as two regression tasks and the same training (402,304) and test (41,184) set sequences were used.

RNA polyadenylation. The data for the RNA polyadenylation task was retrieved from APARENT2 [21]. This dataset was originally derived from Bogard et al. [20] and the same processing as in APARENT2 was applied to make the training data more uniform. It contains 185 bp sequences with randomized proximal polyadenylation signal (PAS) sequences that were tested within 12 diverse 3UTR contexts in an MPRA experiment. The objective is to predict the total isoform proportion of a far-away competing distal PAS. This regression task contains 3.3 million training sequences and 80,000 sequences testing.

RNA degradation. The data for the human and mouse RNA degradation tasks were retrieved from Saluki [19]. This dataset contains processed half-lives for different human and mouse RNA sequences. The cross-validation dataset from fold 0 was used and RNA sequences longer than 12kb were removed. This resulted in 10,377 training and 1,297 testing human sequences, and 10,989 training and 1,374 testing mouse sequences.

Protein tasks. Three different protein tasks related to protein fluorescence, stability and meting point, all predicted from the respective CDS sequence, were retrieved from Boshar et al. [57].

Protein fluorescence: Estimating the fitness landscape of protein variants which are many mutations away from the wildtype sequence is one of the core challenges of protein design. This task evaluates a model's ability to predict log-fluorescence of higher-order mutant green fluorescent protein (GFP) sequences. Original data is from an experimental study of the GFP fitness landscape [85]. Inspired from the TAPE and PEER benchmarks [86, 87], the training set was restricted to amino-acid sequences with three or fewer mutations from parent GFP sequences, while the test set corresponded to all sequences with four or more mutations.

Protein stability: It is important for models trained on diverse sequences to be able to accurately predict a small region of the fitness landscape. This task evaluates how well models predict stability around a small region of high-fitness sequences. Coding sequences and labels were taken from the supplementary material of the original experimental study of G. J. Rocklin et al[88]. Labels indicate a peptide's ability to maintain structure at increasing levels of protease, which serves as a proxy for stability.

Protein melting point: Predicting protein melting point can be a challenging task as even single residue mutations can have large impacts [89]. Melting point prediction is a sequence-level regression task that evaluates a model's ability to predict a measure of melting temperature. The same “mixed” splits as described in FLIP [22], which seek to avoid over-emphasis of large clusters, were used. Sequences are clustered at 20% identity with 80% of clusters assigned to the train dataset and 20% of clusters assigned to the test dataset.

### EQUIVALENTS

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### CERTAIN REFERENCES

[1]H. G. S. Consortium, “Initial sequencing and analysis of the human genome,” Nature, vol. 409, no. 6822, pp. 860-921, 2001.
[2]G. Eraslan, Z. Avsec, J. Gagneur, and F. J. Theis, “Deep learning: new computational modelling techniques for genomics,” Nature Reviews Genetics, vol. 20, no. 7, pp. 389-403, 2019.
[3]T. Yue, Y. Wang, L. Zhang, C. Gu, H. Xue, W. Wang, Q. Lyu, and Y. Dun, “Deep learning for genomics: A concise overview,” arXiv preprint arXiv:1802.00810, 2018.
[4]T. Yue, Y. Wang, L. Zhang, C. Gu, H. Xue, W. Wang, Q. Lyu, and Y. Dun, “Deep learning for genomics: From early neural nets to modem large language models,” International Journal of Molecular Sciences, vol. 24, no. 21, p. 15858, 2023.
[5]B. Alipanahi, A. Delong, M. T. Weirauch, and B. J. Frey, “Predicting the sequence specificities of dna- and rna-binding proteins by deep learning,” Nature Biotechnology, vol. 33, p. 831-838, 2015.
[6]Z. Avsec, M. Weilert, A. Shrikumar, S. Krueger, A. Alexandari, K. Dalal, R. Fropf, C. McAnany, J. Gagneur, A. Kundaje, et al., “Base-resolution models of transcription-factor binding reveal soft motif syntax,” Nature Genetics, vol. 53, no. 3, pp. 354-366, 2021.
[7]C. Angermueller, H. J. Lee, W. Reik, and O. Stegle, “Deepcpg: accurate prediction of single-cell dna methylation states using deep learning,” Genome Biology, vol. 18, no. 67, 2017.
[8]D. R. Kelley, J. Snoek, and J. Rinn, “Basset: Learning the regulatory code of the accessible genome with deep convolutional neural networks,” Genomics Research, vol. 26, pp. 990-999, 2016.
[9]K. M. Chen, A. K. Wong, O. G. Troyanskaya, and J. Zhou, “A sequence-based global map of regulatory activity for deciphering human genetics,” Nature Genetics, vol. 54, no. 7, pp. 940-949, 2022. [10]Z. Avsec, V. Agarwal, D. Visentin, J. R. Ledsam, A. Grabska-Barwinska, K. R. Taylor, Y. Assael, J. Jumper, P. Kohli, and D. R. Kelley, “Effective gene expression prediction from sequence by integrating long-range interactions,” Nature Methods, vol. 18, no. 10, pp. 1196-1203, 2021.
[11]B. P. de Almeida, F. Reiter, M. Pagani, and A. Stark, “Deepstarr predicts enhancer activity from dna sequence and enables the de novo design of synthetic enhancers,” Nature Genetics, vol. 54, no. 5, pp. 613-624, 2022.
[12]G. Fudenberg, D. R. Kelley, and K. S. Pollard, “Predicting 3d genome folding from dna sequence with akita,” Nature Methods, vol. 17, p. 1111-1117, 2020.
[13]R. Schwessinger, M. Gosden, D. Downes, R. C. Brown, A. M. Oudelaar, J. Telenius, Y. W. Teh, G. Lunter, and J. R. Hughes, “Deepc: predicting 3d genome folding using megabase-scale transfer learning,” Nature Methods, vol. 17, p. 1118-1124, 2020.
[14]J. Zhou, “Sequence-based modeling of three-dimensional genome architecture from kilobase to chromosome scale,” Nature Genetics, vol. 54, p. 725-734, 2022.
[15]K. Jaganathan, S. K. Panagiotopoulou, J. F. McRae, S. F. Darbandi, D. Knowles, Y. I. Li, J. A. Kosmicki, J. Arbelaez, W. Cui, G. B. Schwartz, et al., “Predicting splicing from primary sequence with deep learning,” Cell, vol. 176, no. 3, pp. 535-548, 2019.
[16]N. Scalzitti, A. Kress, R. Orhand, T. Weber, L. Moulinier, A. Jeannin-Girardon, P. Collet, O. Poch, and J. D. Thompson, “Spliceator: Multi-species splice site prediction using convolutional neural networks,” BMC Bioinformatics, vol. 22, no. 1, pp. 1-26, 2021.
[17]D. R. Kelley, Y. A. Reshef, M. Bileschi, D. Belanger, C. Y. McLean, and J. Snoek, “Sequential regulatory activity prediction across chromosomes with convolutional neural networks,” Genome Research, vol. 28, pp. 739-750, March 2018.
[18]V. Agarwal and J. Shendure, “Predicting mRNA abundance directly from genomic sequence using deep convolutional neural networks,” Cell Reports, vol. 31, p. 107663, May 2020.
[19]V. Agarwal and D. R. Kelley, “The genetic and biochemical determinants of mrna degradation rates in mammals,” Genome Biology, vol. 23, p. 245, 2022.
[20]N. Bogard, J. Linder, A. B. Rosenberg, and G. Seelig, “A deep neural network for predicting and engineering alternative polyadenylation,” Cell, vol. 178, pp. 91-106, 2019.
[21]J. Linder, S. E. Koplik, A. Kundaje, and G. Seelig, “Deciphering the impact of genetic variation on human polyadenylation using aparent2,” Genome Biology, vol. 23, p. 232, 2022.
[22]C. Dallago, J. Mou, K. E. Johnston, B. J. Wittmann, N. Bhattacharya, S. Goldman, A. Madani, and K. K. Yang, “Flip: Benchmark tasks in fitness landscape inference for proteins,” bioRxiv, 2021.
[23]J. Devlin, M.-W. Chang, K. Lee, and K. Toutanova, “Bert: Pre-training of deep bidirectional transformers for language understanding,” arXiv preprint arXiv:1810.04805, 2018.
[24]A. Radford, J. Wu, R. Child, D. Luan, D. Amodei, I. Sutskever, et al., “Language models are unsupervised multitask learners,” OpenAI blog, vol. 1, no. 8, p. 9, 2019.
[25]T. Brown, B. Mann, N. Ryder, M. Subbiah, J. D. Kaplan, P. Dhariwal, A. Neelakantan, P. Shyam, G. Sastry, A. Askell, et al., “Language models are few-shot learners,” Advances in neural information processing systems, vol. 33, pp. 1877-1901, 2020.
[26]H. Dalla-Torre, L. Gonzalez, J. Mendoza-Revilla, N. L. Carranza, A. H. Grzywaczewski, F. Oteri, C. Dallago, E. Trop, B. P. de Almeida, H. Sirelkhatim, G. Richard, M. Skwark, K. Beguir, M. Lopez, and T. Pierrot, “The nucleotide transformer: Building and evaluating robust foundation models for human genomics,” bioRxiv, 2023.
[27]Y. Ji, Z. Zhou, H. Liu, and R. V. Davuluri, “Dnabert: pre-trained bidirectional encoder representations from transformers model for dna-language in genome,” Bioinformatics, vol. 37, no. 15, pp. 2112-2120, 2021.
[28]Z. Zhou, Y. Ji, W. Li, P. Dutta, R. Davuluri, and H. Liu, “Dnabert-2: Efficient foundation model and benchmark for multi-species genome,” arXiv preprint arXiv:2306.15006, 2023.
[29]E. Nguyen, M. Poli, M. Faizi, A. Thomas, C. Birch-Sykes, M. Wornow, A. Patel, C. Rabideau, S. Massaroli, Y. Bengio, et al., “Hyenadna: Long-range genomic sequence modeling at single nucleotide resolution,” arXiv preprint arXiv:2306.15794, 2023.
[30]G. Benegas, S. S. Batra, and Y. S. Song, “Dna language models are powerful zero-shot predictors of non-coding variant effects,” bioRxiv, pp. 2022-08, 2022.
[31]V. Fishman, Y. Kuratov, M. Petrov, A. Shmelev, D. Shepelin, N. Chekanov, O. Kardymon, and M. Burtsev, “Gena-lm: A family of open-source foundational models for long dna sequences,” bioRxiv, pp. 2023-06, 2023.
[32]B. P. de Almeida, H. Dalla-Torre, G. Richard, C. Blum, L. Hexemer, M. G'elard, J. Mendoza-Revilla, P. Pandey, S. Laurent, M. Lopez, A. Laterre, M. Lang, U. S, ahin, K. Beguir, and T. Pierrot, “Segmentnt: annotating the genome at single-nucleotide resolution with dna foundation models,” bioRxiv, 2024.
[33]H. Bao, L. Dong, S. Piao, and F. Wei, “Beit: Bert pre-training of image transformers,”2022.
[34]S. Gidaris, P. Singh, and N. Komodakis, “Unsupervised representation learning by predicting image rotations,” in International Conference on Learning Representations, 2018.
[35]A. Radford, J. W. Kim, C. Hallacy, A. Ramesh, G. Goh, S. Agarwal, G. Sastry, A. Askell, P. Mishkin, J. Clark, G. Krueger, and I. Sutskever, “Learning transferable visual models from natural language supervision,” 2021.
[36]C. Raffel, N. Shazeer, A. Roberts, K. Lee, S. Narang, M. Matena, Y. Zhou, W. Li, and P. J. Liu, “Exploring the limits of transfer learning with a unified text-to-text transformer,” The Journal of Machine Learning Research, vol. 21, no. 1, pp. 5485-5551, 2020.
[37]A. Roberts, H. W. Chung, A. Levskaya, G. Mishra, J. Bradbury, D. Andor, S. Narang, B. Lester, C. Gaffney, A. Mohiuddin, C. Hawthorne, A. Lewkowycz, A. Salcianu, M. van Zee, J. Austin, S. Goodman, L. B. Soares, H. Hu, S. Tsvyashchenko, A. Chowdhery, J. Bastings, J. Bulian, X. Garcia, J. Ni, A. Chen, K. Kenealy, J. H. Clark, S. Lee, D. Garrette, J. Lee-Thorp, C. Raffel, N. Shazeer, M. Ritter, M. Bosma, A. Passos, J. Maitin-Shepard, N. Fiedel, M. Omernick, B. Saeta, R. Sepassi, A. Spiridonov, J. Newlan, and A. Gesmundo, “Scaling up models and data with t5x and seqio,” 2022.
[38] OpenAI, “Chatgpt,” 2023. https://openai.com/blog/chatgpt/.
[39] OpenAI, “Gpt-4 technical report,” 2023.
[40]H. Liu, C. Li, Q. Wu, and Y. J. Lee, “Visual instruction tuning,” arXiv preprint arXiv:2304.08485, 2023.
[41]J.-B. Alayrac, J. Donahue, P. Luc, A. Miech, I. Barr, Y. Hasson, K. Lenc, A. Mensch, K. Millican, M. Reynolds, et al., “Flamingo: a visual language model for few-shot learning,” Advances in Neural Information Processing Systems, vol. 35, pp. 23716-23736, 2022.
[42]S. Huang, L. Dong, W. Wang, Y. Hao, S. Singhal, S. Ma, T. Lv, L. Cui, O. K. Mohammed, Q. Liu, et al., “Language is not all you need: Aligning perception with language models,” arXiv preprint arXiv:2302.14045, 2023.
[43]D. Zhu, J. Chen, X. Shen, X. Li, and M. Elhoseiny, “Minigpt-4: Enhancing vision-language understanding with advanced large language models,” arXiv preprint arXiv:2304.10592, 2023.
[44]C. Li, C. Wong, S. Zhang, N. Usuyama, H. Liu, J. Yang, T. Naumann, H. Poon, and J. Gao, “Llavamed: Training a large language-and-vision assistant for biomedicine in one day,” arXiv preprint arXiv:2306.00890, 2023.
[45]Z. Huang, F. Bianchi, M. Yuksekgonul, T. J. Montine, and J. Zou, “A visual-language foundation model for pathology image analysis using medical twitter,” Nature Medicine, vol. 29, p. 2307-2316, 2023.
[46]M. Y. Lu, B. Chen, D. F. K. Williamson, R. J. Chen, I. Liang, T. Ding, G. Jaume, I. Odintsov, L. P. Le, G. Gerber, A. V. Parwani, A. Zhang, and F. Mahmood, “A visual-language foundation model for computational pathology,” Nature Medicine, vol. 30, p. 863-874, 2024.
[47]H. Touvron, T. Lavril, G. Izacard, X. Martinet, M.-A. Lachaux, T. Lacroix, B. Rozi'ere, N. Goyal, E. Hambro, F. Azhar, et al., “Llama: Open and efficient foundation language models,” arXiv preprint arXiv:2302.13971, 2023.
[48]Z. Lin, H. Akin, R. Rao, B. Hie, Z. Zhu, W. Lu, N. Smetanin, R. Verkuil, O. Kabeli, Y. Shmueli, et al., “Evolutionary-scale prediction of atomic-level protein structure with a language model,” Science, vol. 379, no. 6637, pp. 1123-1130, 2023. [49]S. Liu, H. Cheng, H. Liu, H. Zhang, F. Li, T. Ren, X. Zou, J. Yang, H. Su, J. Zhu, et al., “Llava-plus: Learning to use tools for creating multimodal agents,” arXiv preprint arXiv:2311.05437, 2023.
[50]B. Peng, C. Li, P. He, M. Galley, and J. Gao, “Instruction tuning with gpt-4,” arXiv preprint arXiv:2304.03277, 2023.
[51]R. Taori, I. Gulrajani, T. Zhang, Y. Dubois, X. Li, C. Guestrin, P. Liang, and T. B. Hashimoto, “Alpaca: A strong, replicable instruction-following model,” Stanford Center for Research on Foundation Models. https://crfm. stanford. edu/2023/03/13/alpaca. html, vol. 3, no. 6, p. 7, 2023.
[52]H. Touvron, L. Martin, K. Stone, P. Albert, A. Almahairi, Y. Babaei, N. Bashlykov, S. Batra, P. Bhargava, S. Bhosale, et al., “Llama 2: Open foundation and fine-tuned chat models,” arXiv preprint arXiv:2307.09288, 2023.
[53]J. Li, D. Li, S. Savarese, and S. Hoi, “Blip-2: Bootstrapping language-image pre-training with frozen image encoders and large language models,” 2023.
[54]F. I. Marin, F. Teufel, M. Hofrender, D. Madsen, D. Pultz, O. Winther, and W. Boomsma, “Bend: Benchmarking dna language models on biologically meaningful tasks,” arXiv preprint arXiv:2311.12570, 2023.
[55]J. Mendoza-Revilla, E. Trop, L. Gonzalez, M. Roller, H. Dalla-Torre, B. P. de Almeida, G. Richard, J. Caton, N. L. Carranza, M. Skwark, A. Laterre, K. Beguir, T. Pierrot, and M. Lopez, “A foundational large language model for edible plant genomes,” bioRxiv, 2023.
[56]M. Salvatore, M. Horlacher, A. Marsico, O. Winther, and R. Andersson, “Transfer learning identifies sequence determinants of cell-type specific regulatory element accessibility,” Genome Biology, vol. 5, p. lqad026, 2023.
[57]S. Boshar, E. Trop, B. P. de Almeida, and T. Pierrot, “Are genomic language models all you need?Exploring genomic language models on protein downstream tasks,” LLMs4Bio AAAI Workshop 2024, 2024.
[58]R. Nogueira, Z. Jiang, and J. Lin, “Investigating the limitations of transformers with simple arithmetic tasks,” 2021.
[59]D. Hendrycks, C. Burns, S. Kadavath, A. Arora, S. Basart, E. Tang, D. Song, and J. Steinhardt, “Measuring mathematical problem solving with the math dataset,” in Proceedings of the Neural Information Processing Systems Track on Datasets and Benchmarks (J. Vanschoren and S. Yeung, eds.), vol. 1, Curran, 2021.
[60]X. Song, O. Li, C. Lee, B. Yang, D. Peng, S. Perel, and Y. Chen, “Omnipred: Language models as universal regressors,” 2024.
[61]J. Chen, Z. Hu, S. Sun, Q. Tan, Y. W. an Qinze Yu, L. Zong, L. Hong, J. Xiao, T. Shen, I. King, and Y. Li, “Interpretable rna foundation model from unannotated data for highly accurate ma structure and function predictions,” arXiv preprint arXiv:2204.00300, 2022.
[62]M. Akiyama and Y. Sakakibara, “Informative rna base embedding for rna structural alignment and clustering by deep representation learning,” NAR Genomics & Bioinformatics, vol. 4, no. 1, p. lqac012, 2022.
[63]Y. Zhang, M. Lang, J. Jiang, Z. Gao, F. Xu, T. Litfin, K. Chen, J. Singh, X. Huang, G. Song, Y. Tian, J. Zhan, J. Chen, and Y. Zhou, “Multiple sequence alignment-based ma language model and its application to structural inference,” Nucleic Acids Research, vol. 52, no. 1, p. e3, 2023.
[64]S. Li, S. Moayedpour, R. Li, M. Bailey, S. Riahi, M. Miladi, J. Miner, D. Zheng, J. Wang, A. Balsubramani, K. Tran, M. Zacharia, M. Wu, X. Gu, R. Clinton, C. Asquith, J. Skalesk, L. Boeglin, S. Chivukula, A. Dias, F. U. Montoya, V. Agarwal, Z. Bar-Joseph, and S. Jager, “Codonbert: Large language models for mrna design and optimization,” bioRxiv, 2023.
[65]J. Platt et al., “Probabilistic outputs for support vector machines and comparisons to regularized likelihood methods,” Advances in large margin classifiers, vol. 10, no. 3, pp. 61-74, 1999.
[66]T. Schick, J. Dwivedi-Yu, R. Dessì, R. Raileanu, M. Lomeli, L. Zettlemoyer, N. Cancedda, and T. Scialom, “Toolformer: Language models can teach themselves to use tools,” arXiv preprint arXiv:2302.04761, 2023.
[67]Q. Jin, Y. Yang, Q. Chen, and Z. Lu, “Genegpt: Augmenting large language models with domain tools for improved access to biomedical information,” ArXiv, 2023.
[68]N. Brandes, D. Ofer, Y. Peleg, N. Rappoport, and M. Linial, “Proteinbert: a universal deeplearning model of protein sequence and function,” Bioinformatics, vol. 38, no. 8, pp. 2102-2110, 2022.
[69]J. Hoffmann, S. Borgeaud, A. Mensch, E. Buchatskaya, T. Cai, E. Rutherford, D. d. L. Casas, L. A. Hendricks, J. Welbl, A. Clark, et al., “Training compute-optimal large language models,” arXiv preprint arXiv:2203.15556, 2022.
[70]A. Q. Jiang, A. Sablayrolles, A. Mensch, C. Bamford, D. S. Chaplot, D. d. 1. Casas, F. Bressand, G. Lengyel, G. Lample, L. Saulnier, et al., “Mistral 7b,” arXiv preprint arXiv:2310.06825, 2023.
[71]H. W. Chung, L. Hou, S. Longpre, B. Zoph, Y. Tay, W. Fedus, Y. Li, X. Wang, M. Dehghani, S. Brahma, et al., “Scaling instruction-finetuned language models,” arXiv preprint arXiv:2210.11416, 2022.
[72]C. Li, Z. Gan, Z. Yang, J. Yang, L. Li, L. Wang, and J. Gao, “Multimodal foundation models: From specialists to general-purpose assistants,” 2023.
[73] ENCODE, “An integrated encyclopedia of dna elements in the human genome,” Nature, vol. 489, no. 7414, pp. 57-74, 2012. [74] Roadmap Epigenomics, “Integrative analysis of 111 reference human epigenomes,” Nature, vol. 518, no. 7539, pp. 317-330, 2015. [75]E. Perez, S. Huang, F. Song, T. Cai, R. Ring, J. Aslanides, A. Glaese, N. McAleese, and G. Irving, “Red teaming language models with language models,” 2022.
[76]M. Moor, O. Banerjee, Z. S. H. Abad, H. M. Krumholz, J. Leskovec, E. J. Topol, and P. Rajpurkar, “Foundation models for generalist medical artificial intelligence,” Nature, vol. 616, p. 259-265, 2023.
[77]J. Bradbury, R. Frostig, P. Hawkins, M. J. Johnson, C. Leary, D. Maclaurin, G. Necula, A. Paszke, J. VanderPlas, S. Wanderman-Milne, and Q. Zhang, “JAX: composable transformations of Python+NumPy programs,” 2018.
[78]T. Hennigan, T. Cai, T. Norman, L. Martens, and I. Babuschkin, “Haiku: Sonnet for JAX,” 2020.
[79]D. P. Kingma and J. Ba, “Adam: A method for stochastic optimization,” arXiv preprint arXiv:1412.6980, 2014.
[80]F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, and E. Duchesnay, “Scikit-learn: Machine learning in Python,” Journal of Machine Learning Research, vol. 12, pp. 2825-2830, 2011.
[81]T. H. Phaml, D. H. Tran, T. B. Ho, K. Satou, and G. Valiente, “Qualitatively predicting acetylation and methylation areas in dna sequences,” Genome Informatics, vol. 16, no. 2, pp. 3-11, 2005.
[82]M. Oubounyt, Z. Louadi, H. Tayara, and K. T. Chong, “Deepromoter: robust promoter predictor using deep learning,” Frontiers in genetics, vol. 10, p. 286, 2019.
[83]Q. Geng, R. Yang, and L. Zhang, “A deep learning framework for enhancer prediction using word embedding and sequence generation,” Biophysical Chemistry, vol. 286, p. 106822, 2022.
[84]R. Wang, Z. Wang, J. Wang, and S. Li, “Splicefinder: ab initio prediction of splice sites using convolutional neural network,” BMC bioinformatics, vol. 20, no. 23, pp. 1-13, 2019.
[85]K. Sarkisyan, D. Bolotin, M. Meer, D. Usmanova, A. Mishin, G. Sharonov, D. Ivankov, N. Bozhanova, M. Baranov, O. Soylemez, N. Bogatyreva, P. Vlasov, E. Egorov, M. Logacheva, A. Kondrashov, D. Chudakov, E. Putintseva, I. Mamedov, D. Tawfik, K. Lukyanov, and F. Kondrashov, “Local fitness landscape of the green fluorescent protein,” Nature, vol. 533, pp. 397-401, May 2016. [86]R. Rao, N. Bhattacharya, N. Thomas, Y. Duan, X. Chen, J. Canny, P. Abbeel, and Y. S. Song, “Evaluating protein transfer learning with tape,” 2019.
[87]M. Xu, Z. Zhang, J. Lu, Z. Zhu, Y. Zhang, C. Ma, R. Liu, and J. Tang, “Peer: A comprehensive and multi-task benchmark for protein sequence understanding,” 2022.
[88]G. J. Rocklin, T. M. Chidyausiku, I. Goreshnik, A. Ford, S. Houliston, A. Lemak, L. Carter, R. Ravichandran, V. K. Mulligan, A. Chevalier, C. H. Arrowsmith, and D. Baker, “Global analysis of protein folding using massively parallel design, synthesis, and testing,” Science, vol. 357, no. 6347, pp. 168-175, 2017.
[89]M. M. Pinney, D. A. Mokhtari, E. Akiva, F. Yabukarski, D. M. Sanchez, R. Liang, T. Doukov, T. J. Martinez, P. C. Babbitt, and D. Herschlag, “Parallel molecular mechanisms for enzyme temperature adaptation,” Science, vol. 371, no. 6533, 2021.

1. A method for evaluating multiple biological sequence-based tasks via combined natural language and biological sequence-based queries, the method comprising:
(a) receiving and/or accessing, by a processor of a computing device, (i) a natural language prompt and (ii) biological sequence data representing one or more biological sequences for evaluation;
(b) generating, by the processor, using a biological language encoder, one or more biological sequence embeddings based on the biological sequence data;
(c) determining, by the processor, one or more text embeddings based on the natural language prompt;
(d) generating, by the processor, using a natural language decoder, a natural language response based on (i) the one or more text embeddings and (ii) the one or more biological sequence embeddings; and
(e) storing and/or providing, by the processor, the determined natural language response for display and/or further processing.

2. The method of claim 1, wherein the biological sequence data is or comprises deoxyribonucleic acid (DNA) sequence data representing one or more nucleotide sequence(s).

3. The method of claim 1, wherein the biological sequence data is or comprises ribonucleic acid (RNA) sequence data representing one or more RNA sequence(s).

4. The method of claim 1, wherein the biological sequence data is or comprises polypeptide sequence data representing one or more polypeptide sequence(s).

5. The method of claim 1, the wherein the biological sequence data is or comprises one or more sequence representation(s) of a first type and the method comprises converting, by the processor, the one or more sequence representations of the first type to one or more corresponding sequence representations of a second type for use as input to the biological language encoder.

6. The method of claim 5, wherein the biological language encoder model is or has been trained using a training dataset comprising a plurality of example biological sequences of the second type.

7. The method of claim 1, wherein the biological sequence encoder receives, as input, one or more sequences of tokens, each sequence of tokens representing at least a portion of the one or more biological sequences.

8. The method of claim 7, wherein the biological sequence encoder model generates, the one or biological sequence embeddings based on the one or more sequences of tokens received as input.

9. The method of claim 8, wherein the one or more biological sequence embeddings are or comprise one or more sets of biological sequence embedding vectors, each set of biological sequence embedding vectors (i) corresponding to and generated based on a particular sequence of tokens received as input and (ii) comprising for each token of the particular sequence, a corresponding embedding vector.

10. The method of claim 1, comprising:
generating, by the processor, from the one or more biological sequence embeddings, one or more corresponding projected embeddings, wherein the biological sequence embeddings have a first dimensionality and the corresponding projected embeddings have a second dimensionality, different from the first and matching a dimensionality of the one or more text embeddings; and
using the one or more projected embeddings and the one or more text embeddings as input to the natural language decoder.

11. The method of claim 10, comprising using a projection model to generate the one or more projected embeddings, wherein the projection model receives, as input the one or more biological sequence embeddings and generates, as output the one or more corresponding projected embeddings.

12. The method of claim 11, wherein the projection model comprises one or more cross attention layers.

13. The method of claim 11, wherein the projection model receives the one or more text embeddings as input, thereby generating the one or more projected embeddings based on the biological sequence embeddings and the text embeddings.

14. The method of claim 1, wherein the natural language decoder model is or comprises a pre-trained model, having been trained using a training corpus comprising a plurality of natural language text.

15. The method of claim 1,
wherein the natural language prompt comprises one or more positional sequence tags, each identifying a particular one of the one or more biological sequences and a corresponding position within the natural language prompt, and
wherein the method comprises:
inserting the one or more biological sequence embeddings and/or projections thereof within the one or more text embeddings based on their corresponding positions as identified via the one or more positional sequence tags to create a combined embedding; and
using the combined embedding as input to the natural language decoder model.

16. The method of claim 1, comprising:
generating, by the processor, using a trained projection model, from the one or more biological sequence embeddings, one or more corresponding projected embeddings having a dimensionality matching that of the one or more text embeddings, said trained projection model having been trained using a natural language question and answer dataset comprising a plurality of example natural language prompts and corresponding natural language answers; and
using the one or more projected embeddings and the one or more text embeddings as input to the natural language decoder.

17. The method of claim 16, wherein the biological sequence encoder is a pre-trained and subsequently fine-tuned model, having (i) been initially pre-trained in an unsupervised fashion using a biological sequence training dataset comprising a plurality of example biological sequences, and (ii) subsequently, trained in tandem with the projection model, using the natural language question and answer dataset.

18. The method of claim 1, wherein the natural language decoder is a pre-trained model, having been trained using a training corpus comprising a plurality of natural language text.

19. The method of claim 1, comprising:
prior to step (a), causing, by the processor, display of a graphical user interface (GUI) comprising a textual input widget for user entry of free-form text;
at step (a), receiving, by the processor, via the textual input widget, as the natural language prompt; user input of text; and
at step (d), causing, by the processor, display of the determined natural language response.

20. The method of claim 19, wherein the GUI is or comprises a chatbot graphical dialog (i) comprising the textual input widget and (ii) in which the determined natural language response is displayed.

21. A method for evaluating multiple tasks relating to and accommodating one or more biological input modalities via unified natural language-based query and response interface, the method comprising:
(a) receiving and/or accessing, by a processor of a computing device, (i) a natural language prompt and (ii) biological object data representing a biological object for evaluation, wherein the biological object data is a particular one of a set of possible datatypes, each associated with a particular biological object encoder of a multi-modal machine learning model;
(b) determining and selecting, by the processor, a particular biological object encoder associated with the particular datatype of the biological object data, and generating, by the processor, using the selected biological object encoder, one or more biological object embeddings based on the biological object data;
(c) determining, by the processor, one or more text embeddings based on the natural language prompt;
(d) generating, by the processor, using a natural language decoder, a natural language response based on (i) the one or more text embeddings and (ii) the one or more biological objecting embeddings; and
(e) storing and/or providing, by the processor, the determined natural language response for display and/or further processing.

22. The method of claim 21, wherein the set of possible datatypes comprises one or more types of biological sequence data, each corresponding to and representing a particular type of biological sequence and the multi-modal machine learning model comprises at least one biological language encoder having been trained via a biological sequence training dataset comprising a plurality of example biological sequences.

23. The method of claim 22, wherein the multi-modal machine learning model comprises a multi-omic biological language encoder having been trained via a biological sequence training dataset comprising a plurality of example biological sequences of at least two distinct types.

24. The method of claim 22, wherein the multi-modal machine learning model comprises a plurality of biological language models, each corresponding to a particular type of biological sequence and having been trained on a dataset comprising a plurality of sequences of the corresponding type.

25. The method of claim 21, wherein the set of possible datatypes comprises one or more types of biological structure models representing 3D structure of biological molecules.

26. A system for evaluating multiple biological sequence-based tasks via combined natural language and biological sequence-based queries, the system comprising:
a processor of a computing device; and
memory having instructions stored thereon, wherein the instructions, when executed by the processor, cause the processor to:
(a) receive and/or access (i) a natural language prompt and (ii) biological sequence data representing one or more biological sequences for evaluation;
(b) generate, using a biological language encoder, one or more biological sequence embeddings based on the biological sequence data;
(c) determine one or more text embeddings based on the natural language prompt;
(d) generate, using a natural language decoder, a natural language response based on (i) the one or more text embeddings and (ii) the one or more biological sequence embeddings; and
(e) store and/or provide the determined natural language response for display and/or further processing.

27. A system for evaluating multiple tasks relating to and accommodating one or more biological input modalities via unified natural language-based query and response interface, the system comprising:
a processor of a computing device; and
memory having instructions stored thereon, wherein the instructions, when executed by the processor, cause the processor to:
(a) receive and/or access (i) a natural language prompt and (ii) biological object data representing a biological object for evaluation, wherein the biological object data is a particular one of a set of possible datatypes, each associated with a particular biological object encoder of a multi-modal machine learning model;
(b) determine and/or select a particular biological object encoder associated with the particular datatype of the biological object data, and generate, using the selected biological object encoder, one or more biological object embeddings based on the biological object data;
(c) determine one or more text embeddings based on the natural language prompt;
(d) generate, using a natural language decoder, a natural language response based on (i) the one or more text embeddings and (ii) the one or more biological objecting embeddings; and
(e) store and/or provide the determined natural language response for display and/or further processing.

28-55. (canceled)
